(19) United States
(12) Patent Application Publication
(10) Pub. No.: US 2008/0193387 A1
(43) Pub. Date: Aug. 14, 2008

(54) ESSENTIAL OIL COMPOSITIONS FOR KILLING OR REPELLING ECTOPARASITES AND PESTS AND METHODS FOR USE THEREOF

(76) Inventor: RICKI DE WOLFF, La Jolla, CA (US)

Correspondence Address: RICKI de WOLFF, 8279 Sugarman Drive, La Jolla, CA, 92037, US

(21) Appl. No.: 11/674,882

(22) Filed: Feb. 14, 2007

(51) Int. Cl. A61K 36/85; A61K 36/23; A61K 36/28; A61K 36/53; A61K 36/899; B65D 83/14; C12Q 1/00; C12P 19/28; A61P 33/14; A61K 36/54; A61K 36/61; A61K 36/752

(52) U.S. Cl. 424/47; 222/635; 424/736; 424/739; 424/742; 424/745; 424/750; 424/764; 424/773; 424/774; 435/4; 435/85

(57) ABSTRACT

Essential-oil compositions comprising Lippia javanica essential oil in combination with one, two, three, four, five, six or more essential oils are provided. The Lippia javanica essential-oil compositions are effective for killing and/or repelling ectoparasites and/or pests, including lice, ticks, mosquitoes, mites, ants and fleas. Methods of using the compositions comprising Lippia javanica essential oil in combination with one, two, three, four, five, six or more essential oils for killing or repelling ectoparasites and/or pests also are provided. Also provided are articles of manufacture and kits that include the Lippia javanica essential-oil compositions.

### FIELD OF THE INVENTION

The present invention relates, in general, to pesticidal compositions and, in particular, pesticidal compositions comprising Lippia javanica essential oil in combination with one or more other plant essential oils. In particular, the present invention relates to a formulation comprising Lippia javanica essential oil in combination with one or more other plant essential oils effective as an insecticide and as an insect or pest repellent that is safe and effective, and methods of use thereof.

### BACKGROUND OF THE INVENTION

Ectoparasites, e.g., biting insects, true bugs, flies, fleas, spiders, ticks, mites and lice, are annoying to humans and can be vectors for disease. Infection with ectoparasites in humans is common and remains a major health problem in the U.S. and worldwide. The most common ectoparasites include head lice, body lice, pubic lice and scabies. Mosquitoes also are considered a major vector for transmissible diseases, especially in developing countries.

Synthetic chemical pesticides have been used as means of pest control. For example, one prior approach involves the use of complex, organic insecticides, such as those disclosed in U.S. Pat. Nos. 4,376,784 and 4,308,279. Use of inorganic salts as components of pesticides also has been described (e.g., see U.S. Pat. Nos. 2,423,284 and 4,948,013).

Current compositions and methods for the treatment and repelling of ectoparasites, e.g., insects, true bugs, flies, fleas, spiders, ticks, mites and lice use insecticidal compositions that include synthetic compounds including carbamates, organophosphates such as malathione, chlorinated hydrocarbons such as lindane, synthetic pyrethroids such as permethrin, N,N-diethyl-meta-toluamide (DEET) and ivermectin. Such compositions are available as prescription formulations or over-the-counter formulations. These treatments generally take a topical approach to treatment.

However, increasing numbers of ectoparasites are developing resistance to several of the insecticides in use (e.g., see Thullner, “Impact of pesticide resistance and network for global pesticide resistance management based on a regional structure,” World Animal Revue, Issue 89 (1997) and “Pesticides and Their Application for the Control of Vectors and Pests of Public Health Importance,” World Health Organization, 6^{th} edition (2006)). As the number of species of ectoparasites resistant to these insecticides increases, the effectiveness of these compounds as a therapeutic treatment regime decreases over time.

Further, some of these chemicals are suspected to be carcinogenic or to accumulate in the environment. The public is rejecting the use of chemicals that persist in food, ground water and the environment, and that are toxic, carcinogenic or otherwise incompatible to humans, domestic animals and/or fish.

In addition, application of pesticides to the skin, or to the scalps of school children, can have detrimental side effects. For example, lindane can cause headache, nausea, vomiting, dizziness and seizures, irritability, restlessness, muscle weakness and twitching, convulsions, reproductive damage, anemia, arrhythmia, liver damage, kidney damage and coma (New Jersey Department of Health and Senior Services Hazardous Substance Fact Sheet for Lindane, CAS No. 58-89-9, RTK Substance No. 1117 (2001)). On Mar. 28, 2003, the Food and Drug Administration (FDA) issued a public health advisory concerning the use of topical formulations of Lindane Lotion and Lindane Shampoo for the treatment of scabies and lice. Lindane is banned in the state of California. Pyrethrin can cause pruritis, erythema, dermatitis, papules, paresthesias (abnormal skin sensations), rhinorrhea, sneezing, asthma, headache, nausea and vomiting (e.g., see U.S. Department of Labor, Chemical Sampling Information—Pyrethrin (2006)). Thus, there is a need for a composition and method of treating ectoparasites that does not involve application of such pesticides to the skin or to the scalps of children.

As an alternative to synthetic pesticides such as carbamates, organophosphates and chlorinated hydrocarbons, natural pesticides derived from botanical sources are of great interest. These natural pesticides are considered safe to humans and the environment. Historically, botanical pesticides, such as pyrethrins, camphor and turpentine, have a long history of use. Pyrethroids and pyrethrins continue to enjoy widespread use. However, there are reports that some pests are developing a resistance to pyrethrin. For example, Hunter et al. reports that head lice in Australia were less susceptible to pyrethrums and pyrethrin than a reference strain (Hunter et al., Parasitol. Res 90(6): 476-478 (2003)). Speare et al. describes the resistance of pubic lice to pyrethrin (Speare et al., Australian Fam Physician 30(6): 572-574 (2001)). There also are reports that flies have developed resistance to pyrethroids, such as the housefly Musca domestica (e.g., see Kristensen et al., Pest Manag Sci 57(1): 82-89 (2001)) and the filth fly (e.g., see Meyer et al., Poult Sci 69(5): 736-740 (1990)). McAbee et al. reports that mosquitoes in California have developed a resistance to the pyrethroids permethrin, deltamethrin and pyrethrum (McAbee et al., Pest Manag Sci 60(4): 359-368 (2004)).

Accordingly, there is a need for pesticidal compositions for killing and/or repelling ectoparasites and/or pests containing no pyrethrins, synthetic pyrethroids, chlorinated hydrocarbons, organophosphates, or carbamates, that are effective, safe and that require a short application time. In addition, there is a need for methods for using such compositions that are safe to humans and the environment and relatively inexpensive to use in obtaining acceptable levels of ectoparasite or pest control.

### SUMMARY OF THE INVENTION

The compositions and methods provided herein satisfy these as well as other needs.

It has been discovered that the combination Lippia javanica oil with one or more essential oils is a very good repellent of ectoparasites and pests and is very effective in killing ectoparasites and pests.

The present invention provides a composition and methods for repelling and/or killing pests and ectoparasites. The composition comprises essential oil from Lippia javanica combined with one, two, three, four, five, six or more essential oils. The composition also optionally comprises one or more alcohols, non-essential oils, carrier, emollient, emulsifier or combinations thereof. It has been discovered that Lippia javanica oil in combination with other essential oils is highly effective in repelling and killing ectoparasites and pests. The compositions provided herein contain Lippia javanica oil in combination with other essential oils and do not include harsh pesticidal chemicals, are environmentally friendly, and can be applied topically to humans or animals. Data below show that the compositions comprising Lippia javanica oil are highly effective, e.g., exhibit increased toxicity and faster activity in killing head lice compared to the individual plant essential oils alone.

A primary object of the present invention is to provide novel pesticidal compositions that contain mixtures of essential oil from Lippia javanica in combination with other plant essential oils and/or their constituents.

Another object of the present invention is to provide pesticidal compositions containing combinations, mixtures or blends of essential oil from Lippia javanica in combination with other plant essential oils and/or their constituents.

A further object of the present invention is to provide a safe, non-toxic pesticidal composition based on Lippia javanica essential oil in combination with other plant essential oils and methods for killing and/or repelling pests and/or ectoparasites that will not harm the environment.

Another object of the present invention is to provide pesticidal compositions and methods for treating, controlling, repelling, eradicating and/or killing pests and ectoparasites, e.g., insects (Insecta), true bugs (Hemiptera), flies (Diptera), fleas (Siphonaptera), spiders (Arachnida), ticks and mites (Acari) and lice (Phthiraptera). A particular objective of the present invention is to provide pesticidal compositions comprising Lippia javanica essential oil in combination with other essential oils and methods for the eradication of head lice, body lice, pubic lice, chiggers and scabies in a mammal. Another object of the present invention is to provide pesticidal compositions comprising Lippia javanica essential oil in combination with other essential oils and methods for repelling or killing pests, such as flies, midges, mosquitoes, gnats, and no-see-ums.

The methods provided herein comprise contacting a subject in need of such treatment with a composition that comprises essential oil from Lippia javanica and one, two, three, four, five, six or more essential oils.

A still further object of the present invention is to provide a method for controlling pests by the application of pesticidally effective amounts of a pesticidal composition comprising Lippia javanica essential oil in combination with other essential oils to a locus where such pest control is desired.

Another object of the present invention is to provide a pesticidal composition comprising Lippia javanica essential oil in combination with other essential oils that has a pleasant scent and that can be applied without burdensome safety precautions.

Still another object of the present invention is to provide a pesticidal composition comprising Lippia javanica essential oil in combination with other essential oils that can be inexpensively produced or employed. Yet another object of the present invention is to provide a pesticidal composition comprising Lippia javanica essential oil in combination with other essential oils and methods to which ectoparasites and pests cannot build resistance.

The above and other objectives are accomplished by the present invention which is directed to a pesticidal composition comprising essential oil from Lippia javanica and at least one other plant essential oil, derivatives thereof, and/or their constituents. In addition, the present invention is directed to methods for treating, controlling, repelling, eradicating and/or killing pests and ectoparasites, in the environment or on a subject. In one embodiment, the methods comprise contacting a subject in need of such treatment with a composition comprising essential oil from Lippia javanica and one, two, three, four, five, six or more essential oils. In addition, the present invention is directed to methods for controlling pests by the applying a pesticidally effective amount of the pesticidal compositions provided herein to a locus where pest control is desired. In another embodiment, the methods comprise contacting an ectoparasite or pest with a composition comprising essential oil from Lippia javanica and one, two, three, four, five, six or more essential oils.

In one aspect, provided herein is a topical formulation suitable for application to a subject comprising an amount of a pesticidal composition comprising Lippia javanica essential oil in combination with other essential oils in an amount effective to exhibit pediculicidal, ovicidal and scabicidal activity.

In yet another aspect, provided herein is a method for treating head lice in a mammal, comprising the step of topically applying to the mammal in need thereof, a therapeutically effective amount of a pesticidal composition comprising Lippia javanica essential oil in combination with other essential oils, in an amount exhibiting pediculicidal and ovicidal activity.

In yet another aspect, provided herein is a method for killing ectoparasites and/or pests, comprising the step of topically applying to the ectoparasite or pest an amount of a pesticidal composition comprising Lippia javanica essential oil in combination with other essential oils, in an amount effective to kill the ectoparasite or pest.

In another aspect, provided herein is a method for repelling ectoparasites or pests from a mammal, comprising the step of topically applying to the mammal in need thereof, a therapeutically effective amount of a pesticidal composition comprising Lippia javanica essential oil in combination with other essential oils, in an amount effective to repel ectoparasites and/or pests.

In another aspect, provided herein is a method for repelling ectoparasites or pests, comprising the step of dispersing a composition comprising Lippia javanica essential oil in combination with other essential oils provided herein into the atmosphere. In one embodiment, the essential-oil composition provided herein is dispersed using a diffuser. In another embodiment, a composition provided herein is dispersed in the atmosphere using a pump spray or aerosol spray that produces a fine mist of particles of the essential oil. In one embodiment, the atmosphere is the air in a room of a house, cabin, kennel or barn. In another embodiment, the atmosphere is the area of a porch, veranda, patio or backyard.

In another aspect, provided herein is a method of preparing a composition including an essential oil from Lippia javanica and one, two, three, four, five, six or more essential oils, comprising mixing the essential oils together. In one embodiment, an antioxidant is added to the composition.

In another aspect, provided herein is a method of preparation of a formulation that includes a Lippia javanica essential oil composition as described herein, the method comprising the steps of combining the essential oils, and mixing the essential oils in a fluid carrier. In one embodiment, the fluid carrier is a non-essential oil. In another embodiment, the method includes adding one or more of an alcohol, a preservative, an antioxidant, a skin softening agent, a humectant, an emollient, a surfactant or a moisturizer to the formulation with constant mixing. In embodiments where antioxidants are included, they are added with the essential-oil blend. In one embodiment, the composition includes Lippia javanica essential oil in combination with other essential oils, alcohol and one or more non-essential oils as a carrier fluid, antioxidants, humectants and preservatives. In one embodiment, the method includes liquefying the one or more non-essential oils by heating to 25° C. while stirring; combining the essential oils with thorough mixing in a separate vessel to form a blend of essential oils; combining the non-essential oils and the blend of essential oils with mixing; adding an antioxidant while mixing; mixing any optional skin softening agents, humectants, emollients, surfactants or moisturizers into an alcohol solvent and adding a preservative, such as propylparaben, to the alcohol solvent mixture; and combining the oil and alcohol mixtures.

In another aspect, provided herein is an article of manufacture, comprising a packaging material, within the packaging material an essential oil composition comprising Lippia javanica essential oil in combination with other essential oils as provided herein and formulations thereof, which is effective for repelling or killing an ectoparasite or pest, and a label that indicates that the essential oil composition provided herein or formulation thereof, is used for repelling or killing an ectoparasite or pest. In another embodiment, the article of manufacture includes an essential oil composition comprising Lippia javanica essential oil in combination with other essential oils as provided herein, an applicator and instructions for application. In one embodiment, the applicator is a pump spray. In another embodiment, the applicator is a portable personal aerosol container or spray device, such as described in U.S. Pat. No. 6,415,992.

In another aspect, provided herein are articles of manufacture that comprise containers in which a composition comprising Lippia javanica essential oil in combination with other essential oils is sold and/or distributed. In one embodiment, these containers contain one or more essential-oil formulation(s) comprising Lippia javanica essential oil in combination with other essential oils as provided herein and have instructions for the use of the essential-oil composition. In one embodiment, the containers are metal, glass or plastic (or other appropriate inert material). In one embodiment, the formulation is concentrated. In another embodiment, the formulation is prepared for immediate use. In one embodiment, the instructions pertain to the use of the blend of essential oils comprising Lippia javanica essential oil in combination with other essential oils as provided herein for repelling or killing ectoparasites or pests. In one embodiment, the instructions for use are provided on the outside of the container.

In another embodiment, the present invention also provides kits for treating ectoparasite infestations. In other embodiments, the present invention provides kits for repelling ectoparasites and/or pests. In various embodiments, the kits include a composition comprising Lippia javanica essential oil in combination with other essential oils in a package or other enclosure. In other embodiments, the kits further include a flea comb or a “nit comb” to assist in removing ectoparasites and/or their eggs from hair. In one embodiment, the package is a box that contains one or more compositions provided herein, instructions for use, and optionally an applicator or a comb. In another embodiment, the kit is provided in a wrapping (such as a plastic) that surrounds the kit. In one embodiment, the comb is provided inside the package, but also may be attached to the outside of the package. In other embodiments the kits include a shower cap. In other embodiments the kit comprises instructions that describe how to use the items included in the kit to kill or repel ectoparasites and/or pests.

In another embodiment, the present invention also provides kits for dispensing the essential oil compositions comprising Lippia javanica essential oil in combination with other essential oils. In one embodiment, the kit includes one or more compositions comprising Lippia javanica essential oil in combination with other essential oils, and a pump sprayer for dispensing the composition(s). In another embodiment, the kit includes one or more essential oil compositions provided herein and an applicator for applying the composition(s) to the hair or skin. In another embodiment, the kit includes one or more essential oil compositions provided herein and an applicator for dispensing or applying the composition(s) to ectoparasites or pests.

In another embodiment, the kit includes one or more compositions comprising Lippia javanica essential oil in combination with other essential oils and a diffuser to dispense the composition(s) into the atmosphere. In one embodiment, the kit comprises one or more essential oil compositions provided herein and a pump spray for atomizing or dispersing the composition(s) as a fine spray in the atmosphere. In one embodiment, the kit includes one or more essential oil compositions provided herein and a diffuser, such as any of the diffusers described in U.S. Pat. Nos. 6,645,436, 6,090,349 and 4,762,275. In another embodiment, the kit includes one or more essential oil compositions provided herein and an absorbent carrier for accepting and diffusing the essential oil. In another embodiment, the kit includes one or more essential oil compositions provided herein and a porous diffusing material.

In another embodiment, the kit includes an essential oil composition comprising Lippia javanica essential oil in combination with other essential oils, a testing assay for mites, and instructions for using the provided essential oil composition to treat mite infestations. In one embodiment, the testing assay for mites is an immunoassay, such as described by Mistrello et al. (J Med Entomol. 35(2):143-147 (1998)). In another embodiment, the testing assay for mites is a guanine test or the Acarex™ test kit, sold and distributed by Fisons Pharmaceuticals (Rochester, N.Y.) in the United States (e.g., see Haouichat et al., Indoor and Built Environment 10:109-115 (2001)). The mite test assays generally are “dipstick-type” tests similar to those sold in drugstores for glucose. Such tests generally detect the presence of house dust mite feces.

Additional objects and attendant advantages of the present invention will be set forth, in part, in the description that follows, or may be learned from practicing or using the present invention. The objects and advantages may be realized and attained by means of the instrumentalities and combinations particularly recited in the appended claims. It is to be understood that the foregoing general description and the following detailed description are exemplary and explanatory only and are not to be regarded as limiting in any way.

### DETAILED DESCRIPTION

### A. Definitions

Unless defined otherwise, all technical and scientific terms used herein have the same meaning as is commonly understood by one of skill in the art to which claimed subject matter belongs. All published documents, or portions of documents, cited in the application including, but not limited to, patents, patent applications, articles, books, manuals, and treatises are hereby expressly incorporated by reference in their entirety for any purpose.

In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described.

Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed, e.g., as commonly accomplished in the art or as described herein. The foregoing techniques and procedures can be generally performed using conventional methods well known in the art and as described in various general and more specific references that are cited and discussed throughout the present specification.

As used herein, whenever a numerical range, such as 1-10 or 5% to 50%, appears herein, the range encompasses the entire range bounded by the first and last recited value. For example, “an alkyl of 1 to 20 carbon atoms” means that an alkyl group can contain only 1 carbon atom, 2 carbon atoms, 3 carbon atoms, etc., up to and including 20 carbon atoms. Another example includes “a formulation including 1% to 10% by weight oil,” which means that the formulation includes by weight 1%, 1.1%, 1.2%, 1.3%, 1.4%, 1.5%, 1.6%, 1.7%, 1.8%, 1.9%, 2%, 2.1% . . . , 9.7%, 9.8%, 9.9% or 10% oil.

As used herein, “insecticide” refers to a substance used to prevent, destroy, repel, mitigate, or kill insects.

As used herein, “organophosphate” refers to an organophosphorous compound that displays anti-cholinesterase activity.

As used herein, “malathion” refers to an organophosphate pesticide that has the chemical name S-(1,2-dicarbethoxyethyl)-O,O-dimethyldithiophosphate or ((Dimethoxyphosphinothioyl)thio)butanedioic acid, diethyl ester (CAS No. 121-75-5). U.S. Pat. Nos. 3,352,664, 3,396,223, and 3,515,782 describe the use of malathion in pesticides. The disclosure of these references is incorporated by reference.

As used herein, “mammal” refers to a class of higher vertebrates comprising man and all other animals that nourish their young with milk secreted by mammary glands and that have skin that is more or less covered with hair. Exemplary mammals include humans, monkeys, rodents, sheep, goats, pigs, dairy and beef cattle, dogs, cats, horses, rabbits, guinea pigs and ferrets.

As used herein, “pediculicidal activity” refers to lice killing activity.

As used herein, “ovicidal activity” refers to egg (of lice) killing activity.

As used herein, “ectoparasites” refers to an organism that lives parasitically on the outside of it host. Ectoparasites include insects (Insecta), true bugs (Hemiptera), flies (Diptera), fleas (Siphonaptera), spiders (Arachnida), ticks and mites (Acari) and lice (Phthiraptera).

As used herein, “pests” refers to insects that are detrimental, annoying or a nuisance to plants or animals, including humans or domesticated animals. Exemplary pests include ants, no-see-ums, wall lice, horse lice, chinch bugs, redcoats, bedbugs, chiggers, fleas (including sand fleas, snow fleas and human fleas), drain flies, mites, weevils, aphids, white flies, ants, millipedes, centipedes, carpet beetles, spiders, gnats, fruit flies, cluster flies, phorid flies and black flies.

As used herein, “lice” refers to insects of the order Phthiraptera, which is sometimes split into the order Anoplura, the sucking lice, and the order Mallophaga, the biting lice. All Phthiraptera are wingless external parasites of birds and mammals.

Lice are a parasitic group of the exopterygote insects and are obligate parasites. Lice are permanent ectoparasites of most mammals and they exhibit an unparalleled host specificity and specialization. A particular lice species is generally found on only one host species. Most individuals will spend their entire life cycle on a single host. Lice feed on hair, skin, secretions, and in some species, blood. Eggs are laid on the host, and the immature lice also live on the host.

As used herein, “head lice” refers to Pediculus capitis, which are small parasitic insects exquisitely adapted to living mainly on the scalp and neck hairs of their host. The type of head lice that infests humans is the head louse Pediculus humanus capitis. If separated from their host for 24 hours, head lice will die because they are highly dependent upon human body warmth.

As used herein, “body lice” refers to tiny parasitic insects (Pediculus humanus corporis) that can be spread by close contact with other people. The head louse is generally indistinguishable from the body louse, although head lice are usually smaller than body lice. They feed on human blood, lay eggs and deposit their fecal matter on skin. When body lice are not feeding on blood, they live in the seams and folds of clothing. Body lice can survive if separated from human contact for up to a week without feeding. Infestation with body lice in humans causes intense itching.

As used herein, “pubic lice” refers to the crab louse, Pthirus pubis (Linnaeus), which are small, six-legged creatures that invade the genital hair region, including the pubic and perineal regions, and infest it with their eggs. The pubic louse also may infest the hair of the armpits, beard, mustache, eyebrows and rarely on the eyelids. The pubic louse is relatively immobile when it is on the host, where it usually remains attached and feeding for hours or days at a single spot without removing its mouth parts from the skin.

As used herein, “scabies” refers to an infestation of the skin by the microscopic mite Sarcoptes scabei. Dermatologists estimate that more than 300 million cases of scabies occur worldwide every year. Infestation is common, found worldwide, and affects people of all races and social classes, regardless of personal hygiene. Scabies spreads rapidly under crowded conditions where there is frequent skin-to-skin contact between people, such as in hospitals, institutions, child-care facilities, and nursing homes. Clinical symptoms include severe itching generally caused by an allergic reaction in the patient to the infestation.

As used herein, the term “subject” is an animal, typically a mammal or bird. Included are humans, primates, cattle, pigs, rabbits, goats, sheep, mice, rats, guinea pigs, hamsters, cats, dogs, horses, chickens, ducks, turkeys and others.

As used herein, the term “patient” includes human and animal subjects.

As used herein, “therapeutically effective amount” refers to an amount of drug sufficient to exert the desired therapeutic effect.

As used herein, “sufficient to kill” refers to an amount of a therapeutically effective agent that is sufficient to kill an organism, such as an ectoparasite or pest.

As used herein, “Ovide®” refers to a trade name product having malathion as an active ingredient and is marketed by Taro Pharmaceuticals, NY, U.S.A.

As used herein, “lindane” refers to the generic name of the gamma isomer of hexachlorocyclohexane. It was introduced as a pediculicide and scabicide in 1952 as Kwell® by Reed and Carnrick. Lindane is a persistent, bioaccumulative, and toxic organochlorine insecticide. Lindane is banned in the state of California and in 52 countries. Lindane is suspected of being a carcinogen. A generic lindane shampoo for killing head lice is manufactured by Alpharma, Inc. (New Jersey, U.S.A.).

As used herein, “Nix®” refers to a trade name product having permethrin as an active ingredient. Nix® is marketed by Warner-Lambert, Morris Plains, N.J., U.S.A.

As used herein, the term “carrier” refers to a material, which may be inorganic or organic and of synthetic or natural origin, with which the active compound is mixed or formulated to facilitate its application to a subject, plant, soil, locus or other object to be treated, or its storage, transport and/or handling. In general, any material that may be customarily employed as a carrier in insecticidal formulations are suitable for use with the present invention. The pesticidal compositions provided herein may be employed alone or in the form of mixtures with such solid and/or liquid dispersible carrier vehicles. As used herein, “carriers” include conventional inert pesticide diluents or extenders of the type usable in conventional pesticide formulations or compositions, e.g., conventional pesticide dispersible carrier vehicles such as gases, solutions, emulsions, suspensions, emulsifiable concentrates, spray powders, pastes, soluble powders, dusting agents, granules, foams, pastes, tablets, aerosols, natural and synthetic materials impregnated with active compounds, microcapsules, fumigating cartridges, fumigating cans and fumigating coils, as well as cold mist and warm mist formulations.

Examples of conventional carrier vehicles for use herein include, but are not limited to, aerosol propellants which are gaseous at normal temperatures and pressures, such as propane, butane, isobutene and carbon dioxide; inert dispersible liquid diluent carriers, including inert organic solvents, such as aromatic hydrocarbons, e.g., benzene, toluene, xylene, alkyl naphthalenes, halogenated aromatic hydrocarbons, cycloalkanes, e.g., cyclohexane, paraffins, e.g., petroleum or mineral oil fractions, chlorinated aliphatic hydrocarbons, e.g., methylene chloride, chloroethylenes, alcohols, e.g., methanol, ethanol, propanol, butanol, glycol, as well as ethers and esters thereof, e.g., glycol monomethyl ether, amines, e.g., ethanolamine, amides, e.g., dimethyl formamide, sulfoxides, e.g., dimethyl sulfoxide, acetonitrile, ketones, e.g., acetone, methyl ethyl ketone, methyl isobutyl ketone, cyclohexanone, and/or water, as well as inert dispersible finely divided solid carriers such as ground natural minerals, e.g., kaolins, clays, vermiculite, alumina, silica, chalk, i.e., calcium carbonate, talc, attapulgite, montmorillonite, kieselguhr, and ground synthetic minerals, e.g., highly dispersed silicic acid, and silicates, e.g., alkali silicates.

As used herein, the term “essential oil” means a volatile oil derived from the leaves, stem, flower or twigs of plants or synthetically-made compounds that have the same chemical attributes. The essential oil usually carries the odor or flavor of the plant. Chemically, each plant essential oil or derivative thereof, which may be extracted from natural sources or synthetically made, generally contains, as a major constituent, an acyclic monoterpene alcohol or aldehyde, a benzenoid aromatic compound containing at least one oxygenated substituent or side chain, or a monocarbocyclic terpene generally having a six-membered ring bearing one or more oxygenated substituents. As used herein, “essential oil” includes derivatives thereof, including racemic mixtures, enantiomers, diastereomers, hydrates, salts, solvates, metabolites, analogs, and homologs.

Examples of such essential oils or their constituents include, but are not limited to, eucalyptus oil, geranium oil, lemongrass oil, petitgrain oil, rosemary oil, thyme oil (white and red), lavender oil, tea tree oil, Tagete minuta oil, lovage oil, Lippia javanica oil, lemon oil, orange oil, grapefruit oil, oil of bergamot, galbanun oil, synthetic types of organic fragrances as described in U.S. Pat. Nos. 4,411,829 and 4,314,915, acetophenone, allyl caprate, α-amylcinnamic aldehyde, amyl salicylate, trans-anethole, anisaldehyde, benzyl alcohol, benzyl acetate, benzyl propionate, borneol, β-caryophyllene, caryophyllene, cinnamyl acetate, cinnamaldehyde, cinnamic alcohol, cinnamyl alcohol, carvacrol, carveol, citral, citronellal, citronellol, cumin aldehyde, cyclamen aldehyde, decanol, dimethyl salicylate, ethyl butyrate, ethyl caprate, ethyl cinnamate, eucalyptol (cineole), eugenol, iso-eugenol, galaxolide, geranial, geraniol, germacrene D, guaiacol, hexenol, α-hexylcinnamic aldehyde, hydroxycitrolnellal, ionone, ipsdienone, isopropenyl acetophenone, linalol, linalyl acetate, d-limonene, menthol, p-methylacetophenone, methyl anthranilate, methyl dihydrojasmonate, methyl eugenol, methyl ionone, methyl salicylate, neral, α-phellandrene, pennyroyal oil, perillaldehyde, 1- or 2-phenyl ethyl alcohol, 1- or 2-phenyl ethyl propionate, piperonal, piperitenone, piperonyl acetate, piperonyl alcohol, o-isopropenyl anisole, D-pulegone, terpinen-4-ol, terpinyl acetate, 4-tert-butylcyclohexyl acetate, α-terpineol, thymol, trans-tagetenone, myrcenone, linalool, carvone, ipsenone, α-phellandrene, piperitenone, gamma-undecalactone, undecenal, vanillin, and ethyl vanillin.

Essential oils can be pure single compounds, for example, wintergreen oil (methyl salicylate). However, other essential oils are mixtures of compounds, for example turpentine oil (pinene and dipentene); bitter almond oil (benzaldehyde and hydrocyanic acid); lavender oil (including borneol, fenchol, linalol, α-terpineol, terpinen-4-ol, geranyl acetate, linanyl acetate, terpenyl acetate, camphor, 1,8-cineole, camphene, limonene), pinene (3-caryophyllene, farnascene and coumarin); and tea tree oil (including linalol, α-terpineol, terpinen-4-ol, 1,8-cineole, limonene, myrcene, phellandrene, pinene, α-terpene, -caryophyllene and sabinene). Other essential oils, their chemistry and plant families are known in the art. See, for example, S. Price, Aromatherapy Workbook—Understanding Essential Oils from Plant to Bottle, (HarperCollins Publishers, 1993; J. Rose, The Aromatherapy Book—Applications & Inhalations (North Atlantic Books, 1992); and The Merck Index (12th Ed. 1996), each of which is incorporated herein by reference.

When the essential oil is a mixture of compounds, the present invention encompasses each of the constituent compounds of the essential oil. The present invention also encompasses variants or mimics of such compounds that share one or more of their characteristics or functions.

As these plant essential oil compounds are known and used for other uses, they may be routinely prepared by a skilled artisan by employing known methods. Exemplary methods for deriving an essential oil include steam distillation, pressing fruit rinds, solvent extraction, macerating the flowers and leaves in fat and treating the fat with solvent, enfleurage and synthetically. See, e.g., Price, Aromatherapy Workbook—Understanding Essential Oils from Plant to Bottle (HarperCollins Publishers, 1993, the entire disclosure of which is incorporated by reference herein).

As used herein, “non-essential oil” refers to an oil that is not an “essential oil” as defined above. The non-essential oils comprise triglycerides and are generally odorless or have very little odor. Exemplary non-essential oils include olive oil, soybean oil, safflower oil, sunflower oil, almond oil, walnut oil, jojoba oil, macadamia nut oil, coconut oil, palm oil, sesame oil, rapeseed oil, canola oil, rice bran oil, grapeseed oil, flaxseed oil, corn oil, cocoa butter, cottonseed oil and avocado oil. These oils are commercially available (e.g., see J. Rose, The Aromatherapy Book—Applications & Inhalations (North Atlantic Books, 1992); and from Natural Oils, Simi Valley, Calif.; Mountain Rose Herbs, Eugene, Oreg.; and FloraTech, Chandler, Ariz.).

As used herein, “Lippia javanica oil” refers to the oil from a species of the family Verbenaceae, which is a family of herbs and shrubs or small trees that have aromatic leaves. It includes oil from the species Lippia javanica (Burm f.) Spreng. It is known by its common name, fever tree. Lippia javanica oil is also known as zinziba oil. Lippia javanica grows in South Africa, Swaziland, Zambia, Botswana, Kenya, Malawi, Tanzania and Mozambique. The leaves of Lippia javanica give off a strong lemon-like smell when crushed. This plant is acknowledged to have medicinal properties and is widely used throughout South Africa where it is used extensively in traditional herbal preparations. The chemical constituents of Lippia javanica oil include various levels of caryophyllene, β-caryophyllene, carvone, germacrene D, ipsenone, ipsdienone, o-isopropenyl-anisole, limonene, linalool, myrcene, myrcenone, myrcene oxide, (Z)-ocimenone, α-phellandrene, piperitenone and trans-tagetenone (e.g., see Mokoka, “Indigenous Knowledge of Fever Tree (Lippia javanica) and Effect of Shade Netting on Plant Growth, Oil Yield and Compound Composition,” Thesis, University of Pretoria etd. (2005)). Myrcenone, cis-ocimenone and trans-ocimenone are reported to be major constituents of the essential oil of Lippia javanica (e.g., see Mwangi et al., “Essential oils of Kenyan Lippia species. Part III,” Flavour and Fragrance Journal 6(3): 221-224 (1991)). The disclosure of each of these references is incorporated in its entirety by reference herein. The oil is commercially available (e.g., from Liberty Natural Products, Portland, Oreg.; Designed Verve, Somerset West, South Africa; Asia-Pacific Aromatherapy, Central, Hong Kong).

As used herein, “Tagete minuta oil” refers to the oil from the ornamental commonly referred to as marigolds. The Tagete genus (Fam. Compositae) contains some fifty strongly scented herbs that have been used globally as traditional herbal medicines for many years. Tagete oil is produced in Egypt, India, Mexico, South America, Madagascar and South Africa. The chemical constituents of Tagete minuta oil include various levels of β-caryophyllene, dihydrotagetone, limonene, β-ocimene, (Z)- and (E)-ocimenone, piperitone, (Z)- and (E)-tagetone, and (Z)- and (E)-tagetenone. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “Levisticum officinalis oil” refers to oil from the perennial herb commonly referred to as lovage. All parts of the plant are strongly aromatic and contain extractable essential oils. Lovage is native to northern Europe and grows throughout the eastern United States. The chemical constituents of Levisticum officinalis oil include n-butylidene phthalide, n-butyl-phthalide, sedanonic anhydride, d-terpineol, carvacrol and eugenol. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “lemongrass oil” refers to the oil from Cymbopogon citratus, a tropical grass native to Asia. The fragrance is described as being lemony with grassy notes. It is also reminiscent of the scent of citronella. It is cultivated throughout Asia, Sri Lanka and Guatemala. The chemical constituents of lemongrass oil includes mainly citral and also d-limonene and geraniol. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “Lavendula augustifolia oil” refers to oil extracted from the flowering tops of Lavendula augustifolia, also known as Lavendula officinalis and commonly referred to as lavender. The fragrance of lavender oil is a subtle floral in combination with herbaceous notes. The lavender plant is native to the Mediterranean region. The oil is commercially produced in France, Italy, England, Spain and China. The chemical constituents of Lavendula augustifolia oil include linalyl acetate, linalol, geraniol, cineole, limonene, α-terpinenol, β-caryophyllene, farnascene, borneol, fenchol, linalol, terpinen-4-ol, geranyl acetate, terpenyl acetate, camphor, 1,8-cineole, camphene and limonene. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “Melaleuca alternifolia oil” refers to the essential oil from the bottlebrush tree and is commonly called tea tree oil. Melaleuca alternifolia is indigenous to New Zealand and Australia but also grows in Southern California. The oil usually is produced by distillation of the leaves. Melaleuca alternifolia oil has a medicinal odor although some people characterize the smell as a pungent spicy woodsy scent. The chemical constituents of Melaleuca alternifolia oil include various levels of α-pinene, sabinene, α-terpinene, limonene, p-cymene, 1,8-cineole, γ-terpinene, terpinolene, terpinen-4-ol, α-terpineol, aromadendrene, d-cadinene, globulol, and viridiflorol. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “Thymus vulgaris oil” refers to the essential oil commonly called thyme leaf oil and includes the red thyme oil and the white thyme oil. The essential oil is usually produced by distillation of the thyme plants. The first distillation yields “red thyme oil,” which varies in color from red to orange to brown. The first distillation has an intense warm, sweet and spicy smell, although it has been characterized as having medicinal notes. Further distilling removes the constituents that produce the red color, producing white thyme oil, a clear or pale yellow oil with a sweet, fresh and mild green aroma. The chemical constituents of Thymus vulgaris oil include various levels of thymol, borneol, camphene, carvacrol, p-cymene, estragol, geraniol, linalool, terpinene and thuyanol. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “Rosmarinum officinalis oil” refers to the essential oil commonly called rosemary leaf oil. Rosemary is a evergreen Mediterranean shrub with a long history of culinary and medicinal use. The essential oil is usually extracted in the spring from flowering branches, including the needles as well its flowers, which produce large amounts of a sweetly pungent essential oil. The fragrance is described as having an intense, sweet piney, medicinal or eucalyptus aroma. The chemical constituents of Rosmarinum officinalis oil include various levels of borneal, borneol, bornyl acetate, camphene, camphor, cineole, limonene, linalool, octanone, pinenes and terpineol. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “petitgrain oil” refers to the essential oil derived from Citrus aurantium, Citrus var. amara, the bitter orange tree. It also is referred to as petigrain oil. Petitgrain oil is usually produced by steam distillation of the leaves and twigs of the tree and can be extracted from the unripe fruit. Petitgrain oil has a sweet, bright floral orange scent, with a woody undertone. The chemical constituents of petitgrain oil include various levels of citral, geranyl acetate, limonene, linalool, linalyl acetate, nerol, α-pinene, β-pinene, termineol and terpinene. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “oil of bergamot” refers to the essential oil of the citrus tree Citrus bergamia. Oil of bergamot is used to provide the distinctive flavor of Earl Grey tea. Oil of bergamot is also known as bergamot oil, and is usually produced the cold-pressing the peel of the nearly ripe fruit. The aroma of oil of bergamot is sweet, slightly floral orange or lemon citrus fragrance with a warm spicy quality. Bergamot oil can contain as a constituent bergaptene, which can cause burns if used on sensitive skin that has been exposed to sunlight. This constituent can be distilled out of the oil to produce an essential oil that is essentially free of bergaptene. The chemical constituents of Citrus bergamia oil include various levels of α-bergaptene, β-bisabolene, geraniol, geraniol acetate, limonene, linalool, linalyl acetate, myrcene, nerol, neryl acetate, γ-terpinene, α-pinene, β-pinene and terpinene. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “eucalyptus oil” refers to the essential oil from the Eucalypts Eucalyptus globulus and Eucalyptus radiate. The essential oil is generally recovered by steam distillation of the leaves. The chemical constituents include various amounts of 1,8-cineol, α-, β-, and γ-terpinene, β-pinene, terpinen-4-ol, linalool oxide, α-gurjunene, aromadendrene, alloaromadendrene, globulol(10-hydroxyaroma-dendrene (e.g., see Nishimura, J Argic Food Chem 27(2): 432-435 (1979)). Cineole is the major constituent. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “lemon oil” refers to the essential oil of the fruit of the hybrid citrus tree of cultivated origin, Citrus×limon. The lemon is a member of the Rutaceae family. The oil is generally extracted by cold compress of the peel of the fruit. The aroma is fresh and sharp. The chemical constituents include various amounts of limonene, camphene, myrcene, phellandrene, pinene terpene, and azulene. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “orange oil” refers to the essential oil of the fruit of the Citrus aurantium or Citrus vulgaris species of the Rutaceae family. The oil is extracted by cold compress of the peel of the fruit. The aroma is sweet, citrus and floral. The chemical constituents include various amounts of limonene, citral, citronellal, decadienal, α-sinensal, β-sinensal, valencene, tangeretin, sinensetin, nobiletin, hexamethoxyflavone and heptamethoxyflavone. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “grapefruit oil” refers to the essential oil expressed from the rinds of the large fruits from cultivated grapefruit trees (Citrus paradisi Macfad., or Citrus×paradisi). The oil is usually extracted by cold compress of the peel of the fruit. The aroma is characteristic and regarded as a fresh, sweet citrus. The chemical constituents include various amounts of nootkatone, thep-menth-1-en-8-thiol, ethyl butyrate, (Z)-3-hexenal, 1-hepten-3-one, 4-mercapto-4-methyl-2-pentanone, d-limonene, decanal and furanocoumarin (e.g., see Ericson et al., Drug Metab Dispos 25(11): 1228-1233 (1997). The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “cinnamon oil” refers to the essential oil from trees and shrubs of the genus Cinnamomum, generally from Cinnamomum zeylanicum. The essential oil is obtained by steam distillation of the leaves, twigs, waste bark and fruits of the tree. The chemical constituents include various amounts of cinnamic aldehyde, cinnamaldehyde, eugenol, methyleugenol, benzyl benzoate and eugenyl acetate. The oil is commercially available (e.g., see J. Rose, The Aromatherapy Book—Applications & Inhalations (North Atlantic Books, 1992); and from Berje Essential Oils, Bloomfield, N.J.; Liberty Natural Products, Portland, Oreg.; and Mountain Rose Herbs, Eugene, Oreg.).

As used herein, “essential-oil blend as provided herein” refers to a composition comprising Lippia javanica essential oil in combination with one, two, three, four, five, six or more other essential oils.

As used herein, “treatment” means any manner in which one or more of the symptoms of a disease or disorder are ameliorated or otherwise beneficially altered. Treatment also encompasses any use of the compositions herein, such as use for treating, repelling and/or eradicating any ectoparasite or pest.

As used herein, “amelioration” of the symptoms of a particular disorder by administration of a particular composition refers to any lessening, whether permanent or temporary, lasting or transient that can be attributed to or associated with administration of the composition.

As used herein, the term “monitoring” refers to observing an effect or absence of any effect. In certain embodiments, one monitors pests after contacting those pests with a composition provided herein. Examples of effects that can be monitored include, but are not limited to, behavior, knockdown, incapacitation and death.

As used herein, the term “contacting” refers to bringing two or more materials into close enough proximity that they can interact. In certain embodiments, contacting can be accomplished in a vessel such as a test tube or a petri dish. In certain embodiments, contacting can be performed in the presence of additional materials. In certain embodiments, contacting can be accomplished in a room or barn. In certain embodiments, contacting can be accomplished in a field.

As used herein, a “diffuser” or “diffuser” is a structure or a device or combination thereof that dispenses essential oils into the atmosphere. In one embodiment, a diffuser is a structure having a porous diffusing member into which aromatic essential oils are impregnated and a heat source, such as an incandescent lamp, stove top, hot air source such as a heating duct, or heating element, is used to accelerate diffusion of the essential oils impregnated into the diffusing member into the atmosphere. When using the diffuser, the diffusing member saturated or impregnated with the essential oil is heated using a heat source, such as by turning on an incandescent lamp. The heat source, such as an incandescent lamp, accelerates diffusion of the essential oil impregnated into the diffusing member, so that the essential oil may be diffused over the whole room in a short time. In some embodiments, the diffusing member is a plate onto which the essential oil is applied and the application of heat accelerates the diffusion of the essential oil throughout the room. Such embodiments are described in the prior art (e.g., see U.S. Pat. Nos. 4,762,275 and 6,090,349). In other embodiments, the diffuser is a device that includes a highly efficient compact diffuser jet; a reservoir for essential oil (e.g., a bottle containing the essential oil); an oil reservoir base; and glass top with a separate electric air pump and air line tubing provides air from the air pump to the diffuser jet (e.g., see U.S. Pat. No. 6,645,436). In this embodiment, the essential oil is dispersed into the atmosphere without heating the essential oil in the process. The diffuser operates by mixing air with the essential oil to produce an extremely fine mist of essential oil, which is dispersed into the air. Using the diffuser jet, the essential oil is finely vaporized or atomized into the atmosphere, diffusing the essential oils in a short amount of time. In other embodiments, the essential oil compositions provided herein are dispersed in the atmosphere using a pump spray with a nozzle configured to produce a fine spray or mist of the composition. In another embodiment, the essential oil compositions provided herein are dispersed into the atmosphere using an aerosol formulation, with a nozzle or jet configured to produce a fine mist or spray of the composition.

As used herein, a composition refers to any mixture of two or more ingredients. It may be a solution, a suspension, a liquid, a powder, a paste, aqueous, non-aqueous or any combination thereof.

As used herein, a combination refers to any association between two or more items.

As used herein, fluid refers to any composition that can flow. Fluids thus encompass compositions that are in the form of liquids, semi-solids, pastes, solutions, aqueous mixtures, gels, lotions, creams and other such compositions.

Unless otherwise indicated, as expressed in the present specification as well as in the set of claims, % (percentage) refers to % wt/wt.

### B. Compositions

The compositions provided herein comprise a combination of Lippia javanica oil with one, two, three, four, five or more essential oils.

In one embodiment, the composition comprises greater than 2% Lippia javanica oil. In one embodiment, the composition comprises at least 5% Lippia javanica oil. In another embodiment, the composition comprises from about or at 5% to about or at 70% Lippia javanica oil.

In one embodiment, the composition comprises at least 10% essential oil. In another embodiment, the composition comprises from about or at 10% to about or at 65% essential oil. In another embodiment, the composition comprises from about or at 20% to about or at 50% essential oil. In other embodiments, the composition comprises 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% essential oil by weight of the composition.

In one embodiment, the composition comprises greater than 2% Lippia javanica oil in combination with one or more essential oils. In one embodiment, the composition comprises greater than 2% Lippia javanica oil in combination with one or more essential oils selected from among eucalyptus oil, lemongrass oil, petitgrain oil, Rosmarinum officinalis (rosemary) oil, Thymus vulgaris (thyme) oil, Lavendula augustifolia (lavender) oil, Melaleuca alternifolia (tea tree) oil, Tagete minuta (marigold) oil, Levisticum officinalis (lovage) oil, cinnamon oil, lemon oil, orange oil, grapefruit oil and oil of bergamot.

In another embodiment, the composition comprises Lippia javanica oil and Lavendula augustifolia oil. In one embodiment, the composition comprises Lippia javanica oil and Lavendula augustifolia oil in a ratio of 3:2. In another embodiment, the composition comprises greater than 2% Lippia javanica oil in combination with and Lavendula augustifolia oil and one or more essential oils selected from among eucalyptus oil, lemongrass oil, petitgrain oil, Rosmarinum officinalis oil, Thymus vulgaris oil, Melaleuca alternifolia oil, Tagete minuta oil, Levisticum officinalis oil, grapefruit oil and lemon oil.

In another embodiment, the composition comprises Lippia javanica oil, Lavendula augustifolia oil, Melaleuca alternifolia oil and eucalyptus oil. In another embodiment, the composition comprises Lippia javanica oil, Lavendula augustifolia oil, Melaleuca alternifolia oil and eucalyptus oil in a ratio of 3:2:2:3. In another embodiment, the composition comprises Lippia javanica oil, Lavendula augustifolia oil, Melaleuca alternifolia oil and eucalyptus oil in combination with one or more essential oils selected from among lemongrass oil, petitgrain oil, Rosmarinum officinalis oil, Thymus vulgaris oil, Tagete minuta oil, Levisticum officinalis oil, grapefruit oil and lemon oil.

In another embodiment, the composition comprises Lippia javanica oil and eucalyptus oil. In one embodiment, the composition comprises Lippia javanica oil and eucalyptus oil in a ratio of 2:1. In another embodiment, the composition comprises Lippia javanica oil and eucalyptus oil in combination with one or more essential oils selected from among lemongrass oil, petitgrain oil, Rosmarinum officinalis oil, Thymus vulgaris oil, Lavendula augustifolia oil, Melaleuca alternifolia oil, Tagete minuta oil, Levisticum officinalis oil, lemon oil, grapefruit oil and oil of bergamot.

In another embodiment, the composition comprises Lippia javanica oil and lemongrass oil. In one embodiment, the composition comprises Lippia javanica oil and lemongrass oil in a ratio of 1:1. In another embodiment, the composition comprises Lippia javanica oil and lemongrass oil in combination with one or more essential oils selected from among eucalyptus oil, petitgrain oil, Rosmarinum officinalis oil, Thymus vulgaris oil, Lavendula augustifolia oil, Melaleuca alternifolia oil, Tagete minuta oil, Levisticum officinalis oil, cinnamon oil, lemon oil, grapefruit oil and oil of bergamot.

In another embodiment, the composition comprises Lippia javanica oil, lemongrass oil and lemon oil. In one embodiment, the composition comprises Lippia javanica oil, lemongrass oil and lemon oil in a ratio of 2:1:1. In another embodiment, the composition comprises Lippia javanica oil, lemongrass oil and lemon oil in combination with one or more essential oils selected from among eucalyptus oil, petitgrain oil, Rosmarinum officinalis oil, Thymus vulgaris oil, Lavendula augustifolia oil, Melaleuca alternifolia oil, Tagete minuta oil, Levisticum officinalis oil, cinnamon oil, grapefruit oil and oil of bergamot.

In another embodiment, the composition comprises at least 3% Lippia javanica oil in combination with Rosmarinum officinalis oil. In one embodiment, the composition comprises Lippia javanica oil and Rosmarinum officinalis oil in a ratio of 3:1. In another embodiment, the composition comprises Lippia javanica oil and Rosmarinum officinalis oil in combination with one or more essential oils selected from among eucalyptus oil, lemongrass oil, petitgrain oil, Thymus vulgaris (thyme) oil, Lavendula augustifolia (lavender) oil, Melaleuca alternifolia (tea tree) oil, Tagete minuta oil, Levisticum officinalis (lovage) oil, cinnamon oil, lemon oil, grapefruit oil and oil of bergamot.

In another embodiment, the composition comprises Lippia javanica oil, Rosmarinum officinalis oil, Lavendula augustifolia oil and petitgrain oil. In one embodiment, the composition comprises Lippia javanica oil, Rosmarinum officinalis oil, Lavendula augustifolia oil and petitgrain oil in a ratio of 3:2:2:3. In another embodiment, the composition comprises Lippia javanica oil, Rosmarinum officinalis oil, Lavendula augustifolia oil and petitgrain oil in combination with one or more essential oils selected from among eucalyptus oil, lemongrass oil, Thymus vulgaris (thyme) oil, Melaleuca alternifolia (tea tree) oil, Tagete minuta oil, Levisticum officinalis (lovage) oil, cinnamon oil, lemon oil, grapefruit and oil of bergamot.

In another embodiment, the composition comprises at least 3% Lippia javanica oil in combination with Melaleuca alternifolia oil. In one embodiment, the composition comprises Lippia javanica oil and Melaleuca alternifolia oil in a ratio of from about 3:4 to about 4:3. In another embodiment, the composition comprises Lippia javanica oil and Melaleuca alternifolia oil in combination with one or more essential oils selected from among eucalyptus oil, lemongrass oil, petitgrain oil, Rosmarinum officinalis (rosemary) oil, Thymus vulgaris (thyme) oil, Lavendula augustifolia (lavender) oil, Melaleuca alternifolia (tea tree) oil, Tagete minuta oil, Levisticum officinalis (lovage) oil, cinnamon oil, lemon oil, grapefruit oil and oil of bergamot.

In another embodiment, the composition comprises Lippia javanica oil, Rosmarinum officinalis oil and Thymus vulgaris oil. In another embodiment, the composition comprises Lippia javanica oil, Rosmarinum officinalis oil and Thymus vulgaris oil in combination with one or more essential oils selected from among eucalyptus oil, lemongrass oil, petitgrain oil, Lavendula augustifolia oil, Melaleuca alternifolia oil, Tagete minuta oil, Levisticum officinalis oil, cinnamon oil, lemon oil, grapefruit oil and oil of bergamot.

In another embodiment, the composition comprises Lippia javanica oil, Rosmarinum officinalis oil, Thymus vulgaris oil and Lavendula augustifolia oil. In another embodiment, the composition comprises Lippia javanica oil, Rosmarinum officinalis oil, Thymus vulgaris and Lavendula augustifolia oil in combination with one or more essential oils selected from among eucalyptus oil, lemongrass oil, petitgrain oil, Melaleuca alternifolia oil, Tagete minuta oil, Levisticum officinalis oil, cinnamon oil, lemon oil and oil of bergamot.

In another embodiment, the composition comprises Lippia javanica oil, Rosmarinum officinalis oil, Thymus vulgaris oil and Melaleuca alternifolia oil. In another embodiment, the composition comprises Lippia javanica oil, Rosmarinum officinalis oil, Thymus vulgaris and Melaleuca alternifolia oil in combination with one or more essential oils selected from among eucalyptus oil, lemongrass oil, petitgrain oil, Lavendula augustifolia oil, Tagete minuta oil, Levisticum officinalis oil, cinnamon oil, lemon oil, grapefruit oil and oil of bergamot.

In another embodiment, the composition comprises Lippia javanica oil, lemongrass oil, Rosmarinum officinalis oil, Thymus vulgaris oil and Melaleuca alternifolia oil. In another embodiment, the composition comprises Lippia javanica oil, lemongrass oil, Rosmarinum officinalis oil, Thymus vulgaris and Melaleuca alternifolia oil in combination with one or more essential oils selected from among eucalyptus oil, petitgrain oil, Lavendula augustifolia oil, Tagete minuta oil, Levisticum officinalis oil, cinnamon oil, lemon oil, grapefruit oil and oil of bergamot.

In another embodiment, the composition comprises Lippia javanica oil, lemongrass oil, Lavendula augustifolia oil, Rosmarinum officinalis oil, Thymus vulgaris oil and Melaleuca alternifolia oil. In another embodiment, the composition comprises Lippia javanica oil, lemongrass oil, Lavendula augustifolia oil, Rosmarinum officinalis oil, Thymus vulgaris and Melaleuca alternifolia oil in combination with one or more essential oils selected from among eucalyptus oil, petitgrain oil, Tagete minuta oil, Levisticum officinalis oil, cinnamon oil, lemon oil, grapefruit oil and oil of bergamot. In another embodiment, the composition comprises Lippia javanica oil, Lavendula augustifolia oil, Melaleuca alternifolia oil, Rosmarinum officinalis oil, Thymus vulgaris oil, lemongrass oil and an alcohol.

In one embodiment, the composition comprises Lippia javanica oil in combination with Levisticum officinalis (lovage) oil. In one embodiment, the composition comprises Lippia javanica oil in combination with one or more essential oils selected from among eucalyptus oil, lemongrass oil, petitgrain oil, Rosmarinum officinalis oil, Thymus vulgaris oil, Lavendula augustifolia oil, Melaleuca alternifolia oil, Tagete minuta oil, cinnamon oil, lemon oil grapefruit oil and oil of bergamot.

In one embodiment, the composition comprises Lippia javanica oil in combination with Levisticum officinalis oil and petitgrain oil. In one embodiment, the composition comprises Lippia javanica oil and petitgrain oil in combination with one or more essential oils selected from among eucalyptus oil, lemongrass oil, Rosmarinum officinalis oil, Thymus vulgaris oil, Lavendula augustifolia oil, Melaleuca alternifolia oil, Tagete minuta oil, cinnamon oil, lemon oil, grapefruit oil and oil of bergamot.

In one embodiment, the composition comprises Lippia javanica oil in combination with Levisticum officinalis oil, petitgrain oil and Tagete minuta oil. In one embodiment, the composition comprises Lippia javanica oil, Levisticum officinalis oil, petitgrain oil and Tagete minuta oil in a ratio of 4:1:1:1. In one embodiment, the composition comprises Lippia javanica oil, Levisticum officinalis oil, petitgrain oil and Tagete minuta oil in combination with one or more essential oils selected from among eucalyptus oil, lemongrass oil, Rosmarinum officinalis oil, Thymus vulgaris oil, Lavendula augustifolia oil, Melaleuca alternifolia oil, cinnamon oil, lemon oil, grapefruit oil and oil of bergamot.

In another embodiment, the composition comprises Lippia javanica oil, lemongrass oil, Rosmarinum officinalis oil and Lavendula augustifolia oil. In one embodiment, the composition comprises Lippia javanica oil, lemongrass oil, Rosmarinum officinalis oil and Lavendula augustifolia oil in a ratio of about or at 3:3:1:1. In another embodiment, the composition comprises Lippia javanica oil, lemongrass oil, Rosmarinum officinalis oil and Lavendula augustifolia oil in a ratio of about or at 3:3:1:1 in combination with one or more essential oils selected from among eucalyptus oil, petitgrain oil, Thymus vulgaris oil, Melaleuca alternifolia oil, Tagete minuta oil, Levisticum officinalis oil, cinnamon oil, lemon oil, orange oil, grapefruit oil and oil of Bergamot. In one embodiment, the composition comprises Lippia javanica oil, lemongrass oil, Rosmarinum officinalis oil and Lavendula augustifolia oil in a ratio of about or at 3:3:1:1 in combination with Melaleuca alternifolia oil.

In one embodiment, the composition comprises Lippia javanica oil, eucalyptus oil and orange oil. In another embodiment, the composition comprises Lippia javanica oil, lemongrass oil, lemon oil and orange oil. In another embodiment, the composition comprises Lippia javanica oil, Lavendula augustifolia oil, Rosmarinum officinalis oil, petitgrain oil and orange oil. In another embodiment, the composition comprises Lippia javanica oil, Melaleuca alternifolia oil, Lavendula augustifolia oil, orange oil and eucalyptus oil. In another embodiment, the composition comprises Lippia javanica oil, Tagete minuta oil, orange oil and cinnamon oil. In another embodiment, the composition comprises Lippia javanica oil, Tagete minuta oil, orange oil and Levisticum officinalis oil.

In another embodiment, the composition comprises one of several specific pairs of essential oils selected from among lavender oil and tea tree oil; rosemary oil and tea tree oil; rosemary and grapefruit oil; lemongrass oil and tea tree oil; lemongrass and grapefruit oil; petitgrain oil and tea tree oil; lavender oil and rosemary oil; lavender oil and grapefruit oil; lavender oil and lemongrass oil; lavender oil and eucalyptus oil; lavender oil and orange oil; petitgrain oil and grapefruit oil; petitgrain oil and rosemary oil; petitgrain oil and lemongrass oil; petitgrain oil and eucalyptus oil; petitgrain oil and orange oil; and rosemary oil and eucalyptus oil. In each of these pairs, the ratio of the oils within the pair can be from about 1:5 to about 5:1.

In other embodiments, the composition comprises Lippia javanica essential oil in combination with one of several specific triplet combinations of essential oils selected from among lemongrass oil, Lavendula augustifolia and Melaleuca alternifolia oil; lemongrass oil, Lavendula augustifolia and Rosmarinum officinalis oil; lemongrass oil, Lavendula augustifolia and Thymus vulgaris oil; Lavendula augustifolia, Melaleuca alternifolia oil and Rosmarinum officinalis oil; grapefruit oil, Lavendula augustifolia and Melaleuca alternifolia oil; grapefruit oil, Lavendula augustifolia and Rosmarinum officinalis oil; grapefruit oil, Lavendula augustifolia and Thymus vulgaris oil; orange oil, Lavendula augustifolia and Melaleuca alternifolia oil; orange oil, Lavendula augustifolia and Rosmarinum officinalis oil; orange oil, Lavendula augustifolia and Thymus vulgaris oil; lemon oil, Lavendula augustifolia and Melaleuca alternifolia oil; lemon oil, Lavendula augustifolia and Rosmarinum officinalis oil; lemon oil, Lavendula augustifolia and Thymus vulgaris oil; petitgrain oil, Lavendula augustifolia and Melaleuca alternifolia oil; petitgrain oil, Lavendula augustifolia and Rosmarinum officinalis oil; petitgrain oil, Lavendula augustifolia and Thymus vulgaris oil; eucalyptus oil, Lavendula augustifolia and Melaleuca alternifolia oil; eucalyptus oil, Lavendula augustifolia and Rosmarinum officinalis oil; and eucalyptus oil, Lavendula augustifolia and Thymus vulgaris oil.

In some embodiments, the composition comprises a carrier. In one embodiment, the carrier is an inorganic or organic material of synthetic or natural origin, with which the essential oils and other ingredients of the compositions provided herein are mixed or formulated to facilitate its application, or its storage, transport and/or handling. In general, any material that may be customarily employed as a carrier in insecticidal formulations are suitable for use with the present composition.

In one embodiment, the carrier is in the form of solid and/or liquid dispersible carrier vehicles. The pesticidal compositions of the present invention can be formulated or mixed with, if desired, conventional inert pesticide diluents or extenders of the type usable in conventional pesticide formulations or compositions, e.g., conventional pesticide dispersible carrier vehicles such as gases, solutions, emulsions, suspensions, emulsifiable concentrates, spray powders, pastes, soluble powders, dusting agents, granules, foams, pastes, tablets, aerosols, natural and synthetic materials impregnated with active compounds, microcapsules, as well as cold mist and warm mist formulations. One skilled in the art can select the appropriate form of particular dosage preparations for specific application made therefrom, such as solutions, emulsions, suspensions, powders, pastes, and granules which are thus ready for use.

Examples of conventional carriers include, but are not limited to, aerosol propellants that are gaseous at normal temperatures and pressures, such as propane, butane, isobutane, and carbon dioxide; inert dispersible liquid diluent carriers, including inert organic solvents, such as aromatic hydrocarbons, e.g., benzene, toluene, xylene, alkyl naphthalenes, halogenated aromatic hydrocarbons, e.g., chloro-benzenes, cycloalkanes, e.g., cyclohexane, paraffins, e.g., petroleum or mineral oil fractions, chlorinated aliphatic hydrocarbons, e.g., methylene chloride and chloroethylenes, alcohols, e.g., methanol, ethanol, propanol, butanol, glycol, as well as ethers and esters thereof, e.g., glycol monomethyl ether, amines, e.g., ethanolamine, amides, e.g., dimethyl formamide, sulfoxides, e.g., dimethyl sulfoxide, acetonitrile, ketones, e.g., acetone, methyl ethyl ketone, methyl isobutyl ketone, cyclohexanone, and/or water, as well as inert dispersible finely divided solid carriers such as ground natural minerals, e.g., kaolins, clays, vermiculite, alumina, silica, chalk (calcium carbonate), talc, attapulgite, montmorillonite, kieselguhr, and ground synthetic minerals, e.g., highly dispersed silicic acid and silicates.

In one embodiment, the present composition includes a solid carrier, such as powders or granules. In the preparation of powders, dust or granulated formulations, the compositions comprising the essential-oil blend is dispersed in and on an appropriately divided carrier. Dusts are admixtures of one or more compositions provided herein with finely divided solids such as talc, amorphous or fumed silica, attapulgite clay, kaolin, kieselguhr, pyrophyllite, chalk, diatomaceous earths, vermiculite, calcium phosphates, calcium and magnesium carbonates, sulfur, flours, and other organic and inorganic solids which acts as carriers for the pesticide compositions provided herein. These finely divided solids may have an average particle size of less than about 50 microns. A typical dust formulation useful for controlling insects contains 1-20 parts of the composition provided herein and 80-99 parts of diatomaceous earth or vermiculite. Granules may comprise porous or nonporous particles. The granule particles are relatively large, with a diameter of about 400-2500 microns typically. The particles are either impregnated or coated with the pesticidal compositions provided herein. Granules generally contain 0.05-15%, and in some embodiments 0.5-5%, active ingredient as the pesticidally effective amount. Thus, the formulations provided herein optionally comprise solid carriers or diluents such as bentonite, fullers earth, ground natural minerals, such as kaolins, clays, talc, chalk, quartz, attapulgite, montmorillonite or diatomaceous earth, smectite clays, chemically modified magnesium aluminum silicate, organically modified montmorillonite clay, hydrated aluminum silicate, fumed silica, aluminum starch octenyl succinate, vermiculite, and ground synthetic minerals, such as dispersed silicic acid, alumina and silicates, crushed and fractionated natural rocks such as calcite, marble, pumice, sepiolite and dolomite, as well as synthetic granules of inorganic and organic meals, and granules of organic materials such as peanut shell, paper waste, sawdust, coconut shells, corn cobs and tobacco stalks. Adhesives, such as carboxymethyl cellulose, natural and synthetic polymers (such as gum arabic, polyvinyl alcohol and polyvinyl acetate) also may be used in the formulations in the form of powders, dusts or granules.

In one embodiment, the composition comprises a carrier that is a fluid carrier. Exemplary fluid carriers include, but are not limited to, alcohols, glycols, non-essential oils, ethers, ketones, aqueous solutions, organic solvents or combinations thereof. In one embodiment, the composition comprises 0-70% fluid carrier. In one embodiment, the composition comprises 5-60% fluid carrier. In one embodiment, the composition comprises 10-50% fluid carrier. In other embodiments, the composition includes 15-40% fluid carrier. In other embodiments, the composition includes 20-30% fluid carrier. In other embodiments, the composition comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% fluid carrier.

In one embodiment, the compositions comprise a carrier fluid that comprises one or more alcohols, one or more glycols, one or more non-essential oils, one or more ethers, one or more ketones, aqueous solutions, one or more organic solvents or combinations thereof.

In one embodiment, the fluid carrier is an alcohol. Some examples of alcohols for use in the composition include, but are not limited to, methanol, ethanol, propanol, isopropanol, butanol, sec-butanol, tert-butanol, pentanol and its various isomers, hexanol and its various isomers, and mixtures thereof. In some embodiments, the lower alkyl alcohol is selected from among C₁-C₈ alcohols. In some embodiments, the lower alkyl alcohol is ethyl alcohol or isopropyl alcohol. In one embodiment, the carrier fluid comprises ethanol. The carrier fluid comprising one or more alcohols is blended with one or more essential oils. In one embodiment, the ratio of carrier fluid comprising one or more alcohols to essential oil is in the range between about 5:95 and about 95:5. In one embodiment, the ratio of carrier fluid comprising one or more alcohols to essential oil is in the range between about 50:50 and about 90:10. In another embodiment, the ratio of carrier fluid comprising alcohols to essential oil is in the range between about 70:30 to about 80:20.

In one embodiment, the carrier fluid is one or more glycols. Exemplary glycols include, but are not limited to, ethylene glycol, propylene glycol, butylene glycol, trimethylene glycol, glycerol, pinacol, and combinations thereof.

In one embodiment, the carrier fluid is one or more non-essential oils. Non-essential oils suitable as carrier fluids in the compositions include synthetic, refined, and/or natural non-essential oils.

In one embodiment, the carrier fluid is a synthetic non-essential oil. Synthetic non-essential oils include petroleum sources, chemicals or hydrocarbon materials that have been processed.

In one embodiment, the carrier fluid is a refined non-essential oil. Refined non-essential oils are non-essential oils that have been processed, either by distillation or chemical processing, for example, by treatment with an acid. Mineral oil is an example of a refined non-essential oil.

In one embodiment, the carrier fluid is a natural non-essential oil. Natural oils are most often obtained from plants or animals, either directly or through processing. Exemplary non-essential oils include olive oil, babassu oil, soybean oil, safflower oil, sunflower oil and high oleic acid sunflower oil or combinations thereof, almond oil, walnut oil, jojoba oil, macadamia nut oil, coconut oil, palm oil, sesame oil, rapeseed oil, canola oil, rice bran oil, grapeseed oil, flaxseed oil, corn oil, cocoa butter, cottonseed oil and avocado oil and combinations thereof. The natural non-essential oils also include those that are modified to enhance stability. Examples include esters of jojoba oil, such as those sold under the trade name Floraesters™ (FloraTech, Gilvert, Arez.) and PEG-ylated natural non-essential oils sold under the trade name Florasolvs™ (FloraTech, Gilbert, Ariz.).

In one embodiment, a single non-essential oil is used as a carrier fluid. The non-essential oil is selected from among olive oil, babassu oil, soybean oil, safflower oil, sunflower oil and high oleic acid sunflower oil or combinations thereof, almond oil, walnut oil, jojoba oil, macadamia nut oil, coconut oil, palm oil, sesame oil, rapeseed oil, canola oil, rice bran oil, grapeseed oil, flaxseed oil, corn oil, cocoa butter, cottonseed oil and avocado oil. In another embodiment, the carrier fluid is coconut oil. In another embodiment, the carrier fluid is soybean oil.

In another embodiment, a blend of two or more non-essential oils is used as a carrier fluid. In one embodiment, the carrier fluid is a blend of two non-essential oils selected from among olive oil, babassu oil, soybean oil, safflower oil, sunflower oil and high oleic acid sunflower oil or combinations thereof, almond oil, walnut oil, jojoba oil, macadamia nut oil, coconut oil, palm oil, sesame oil, rapeseed oil, canola oil, rice bran oil, grapeseed oil, flaxseed oil, corn oil, cocoa butter, cottonseed oil and avocado oil. In one embodiment, the carrier fluid is a blend of coconut oil and soybean oil. In another embodiment, the carrier fluid is a blend of almond oil and olive oil. In another embodiment, the carrier fluid is coconut oil and olive oil. In another embodiment, the carrier fluid is almond oil, coconut oil and olive oil.

In another embodiment, the carrier fluid is one or more ether. Exemplary ether carrier fluids include, but are not limited to, diethyl ether, n-propyl ether, isopropyl ether, ethylene glycol monomethyl ether, ethylene glycol dimethyl ether, ethylene glycol monoethyl ether, ethylene glycol diethylether, propylene glycol mono-and dimethyl ethers, propylene glycol mono and diethyl ethers, butylene glycol mono and dimethyl ethers, butylene glycol mono and diethyl ethers, and combinations thereof. In some embodiments, the glycol is propylene glycol or hexylene glycol.

In another embodiment, the carrier fluid is one or more ketone. Exemplary ketone carrier fluids include, but are not limited to, acetone, methyl ethyl ketone, methyl propyl ketone, ethyl ketone, methyl isopropyl ketone, benzyl methyl ketone, and combinations thereof.

In another embodiment, the carrier fluid is an aqueous solution. In one embodiment, the aqueous solution comprises a solubilizing compound that helps to solubilize or emulsify the one or more essential oils into the aqueous solution. Exemplary solubilizing compounds suitable to solubilize or emulsify the essential oils in the aqueous solution include, but are not limited to, surfactants, alcohols, ethers and ketones. In some embodiments, the ketone is N-methyl pyrrolidone or acetone.

In one embodiment, the carrier fluid is a combination of these carrier fluids. In one embodiment, the carrier fluid comprises one or more non-essential oils and one or more alcohols. In another embodiment, the carrier fluid comprises an aqueous solution, an alcohol and an emulsifier. Examples of suitable emulsifiers include, without limitation, stearic acid, cetyl alcohol, PEG-100, stearate and glyceryl stearate, cetearyl glucoside, polysorbate 20, ceteareth-20, cetyl alcohol, cetearyl alcohol, self-emulsifying wax (e.g., Lipowax P), isopropyl palmitate, cetyl palmitate, stearyl alcohol, lecithin, hydrogenated lecithin, steareth-2, steareth-20, and polyglyceryl-2 stearate. Other emulsifiers also can be included in the compositions of the present invention without departing from the principles taught herein. Examples of such emulsifiers include those found in McCutcheon's 2002, Volume 1: Emulsifiers & Detergents (North American Edition) (2002). Emulsifiers can be obtained from, for example, Lipo Chemicals, Inc. (Paterson, N.J.), Croda, Inc., Goldschmidt A G (Essen, Germany), and Uniqema, Inc. (New Castle, Del.).

In one embodiment, the carrier fluid comprises one or more non-essential oils, one or more alcohols, and an emulsifying agent. In one embodiment, the carrier fluid is coconut oil, isopropyl palmitate and ethanol. In another embodiment, the carrier fluid is coconut oil, olive oil and lecithin. In another embodiment, the carrier fluid is coconut oil, olive oil, alcohol and lecithin. In another embodiment, the carrier fluid is isopropyl palmitate, coconut oil, alcohol and water. In another embodiment, the carrier fluid is isopropyl palmitate, coconut oil, alcohol and water. In another embodiment, the carrier fluid is isopropyl palmitate, coconut oil, soybean oil and alcohol.

In one embodiment, the carrier fluid forms a solution or an emulsion with the essential-oil blend. In one embodiment, the composition is in the form of an emulsion, prepared by dispersing the compositions provided herein in water with suitable surface-active agents. Surface-active agents suitable for use in the compositions provided herein include, without limitation, emulsifying agents, such as non-ionic and/or anionic emulsifying agents, e.g., sodium dodecyl benzene sulfonate, polyethylene oxide esters of fatty acids, polyethylene oxide ethers of fatty alcohols, alkyl sulfates, alkyl sulfonates, aryl sulfonates, albumin hydrolyzates, alkyl arylpolyglycol ethers, magnesium stearate and sodium oleate.

One skilled in the art can determine an optimal formulation for the carrier fluid to be used with a particular essential oil or blend of essential oils.

In one embodiment, the non-essential oil that is used as the carrier fluid is coconut oil. The coconut oil comprises from about 2% to about 20% of the composition. In one embodiment, the coconut oil comprises 3%, 4%, 5%, 6%, 7%, 8%, 9%, 10%, 11%, 12%, 13%, 14%, 15%, 16%, 17%, 18% or 19% of the formulation. In another embodiment, the carrier fluid comprises a mixture of soybean oil and coconut oil. In one embodiment, the blend of coconut oil and soybean oil comprises from about 2% to about 20% of the composition. In another embodiment, the blend of coconut oil and soybean oil comprises 4%, 5%, 6%, 7%, 8%, 9%, 10%, 11%, 12%, 13%, 14%, 15%, 16%, 17%, 18% or 19% of the composition In the contemplated applications, the compositions provided herein encompass compositions that include a carrier in which the essential oil compositions provided herein are present in an amount between about 0.01-95% by weight. In one embodiment, the essential-oil blend is present in an amount of 0.5 to 90% by weight, of the composition. In one embodiment, the essential-oil blend is present in an amount of 10% to 80% by weight, of the mixture. In another embodiment, the essential-oil blend is present in an amount of about 20% to about 70% by weight, of the mixture.

In one embodiment, the essential-oil blend is added to the carrier in a ratio of about 1:99 to about 50:50. In another embodiment, the ratio of essential-oil blend to carrier is between about 2:98 and about 45:55. In another embodiment, the ratio of essential-oil blend to carrier is about 3:97 to about 40:60. In another embodiment, the ratio is about 4:96 to about 35:65. In another embodiment, the ratio of essential-oil blend to carrier is about 5:95 to about 30:70.

In another embodiment, compositions suitable for direct application to a subject or to the pest or ectoparasite or environment application, such as field, bedding, or clothing, are provided in which the essential-oil blend is present in an amount between about 0.1-90% by weight of the mixture. In one embodiment, the essential oil composition is dispersed in an inert finely divided carrier solid. In another embodiment, the composition provided herein is dispersed in a liquid carrier fluid, optionally including a surface-active agent, such as an emulsifying agent and/or a dispersing agent, and an amount of the active compound which is effective for the purpose in question and which is generally between about 0.0001-95% by weight of the mixture.

In another embodiment, the compositions provided herein include silicone to impart lubrication qualities or viscosity. Suitable silicones that can be used in the compositions provided herein include, without limitation, nonvolatile silicone fluids such as dimethicone copolyol, cyclomethicone, polydimethylsiloxane, cyclic dimethyl polysiloxane, aminosilicones, phenylsilicones, diphenyldimethicones, phenyltri-methicones, cyclopentasiloxane, dimethicone copolyol (a polymer of dimethyl-siloxane with polyoxyethylene and/or polyoxypropylene), dimethicone copolyol, cetyldimethicone copolyol, cetyl dimethicone, cetyl dimethiconecopolyol, and amino-propyl PEG-7 PEG-3 dimethicone copolyol and dimethiconols. Such silicones are readily available from Goldschmidt A G, General Electric (Waterford, N.Y.), and Dow Corning Corporation (Midland, Mich.). The silicone oils are generally selected to have viscosities in the range about 20 to about 10000 mPa·s. In some embodiments, the silicone is selected from among cyclomethicone and dimethicone and silicone elastomers such as dimethicone/vinyl dimethicone crosspolymer, dimethicone crosspolymer, cyclopentasiloxane, polysilcone-11 and those described in Starch, “New Developments in Silicone Elastomers in Skin Care” (2002) and in U.S. Pat. Nos. 5,919,437, 5,919,468, 5,266,321, 5,654,362, 5,412,004 and 5,871,761, the disclosure of each of which is specifically incorporated by reference herein.

In one embodiment, the compositions provided herein comprise, by weight, from 0.001% to about 10.0% silicone. In another embodiment, the composition comprises about or 0.01% to about or 8% silicone by weight. In another embodiment, the composition comprises about or 0.05% to about or 5% silicone by weight.

In another embodiment, the compositions provided herein comprise a humectant and/or an emollient. In one embodiment, the humectant and/or emollient is selected from among alkylmonoglycerides, alkyldiglycerides, triglycerides, derivatives of these oils such as hydrogenated oils, lanolin derivatives, mineral oils or paraffin oils, perhydrosqualane, squalene, diols such as 1,2-propanediol, 1,3-butanediol, cetyl alcohol, stearyl alcohol, oleic alcohol, glycerin, glycol, butylene glycol, polyethylene glycol esters, polyethylene glycols or polypropylene glycols, fatty esters such as isopropyl palmitate, 2-ethylhexyl cocoate, myristyl myristate, isopropyl myristate, isopropyl stearate, glyceryl triacetate, ascorbyl palmitate, octyl dodecanol, hexyl decanol, oleyl alcohol, decyl oleate, hexyl laureate, dioctyl cyclohexane, glycearyl stearate, Ceteareth 20, Cetereath 12, cetyl palmitate, esters of lactic acid, stearic acid, behenic acid, isostearic acid, lecithin, collagen, elastin, keratin, lanolin, polyols such as sorbitol, xylitol and maltitol, polymeric polyols such as polydextrose, mineral oil, petrolatum, paraffin, synthetic alcohols such as benzyl alcohol, butyl alcohol, cetearyl alcohol, cetyl alcohol, myristyl alcohol, diglyceryl caprylate and combinations thereof. In one embodiment, the emollient comprises from about or at 1% to about or at 20% of the composition. In another embodiment, the emollient comprises 1%, 2%, 3%, 4%, 5%, 6%, 7%, 8%, 9%, 10%, 11%, 12%, 13%, 14%, 15%, 16%, 17%, 18%, 19% or 20% of the composition.

In some embodiments, the formulations are preserved by adding preservatives that are commonly known to one skilled in the art. In some embodiments, the preservative is an ester of p-hydroxybenzoic acid. In some embodiments, the preservative is methylparaben, propylparaben, butylparaben, sodium benzoate, potassium benzoate, sorbic acid, 2-phenoxyethanol, methylchloroisothiazolinone, methylisothiazolinone, hexylene glycol, chlorphenesin or combinations thereof.

In one embodiment, the compositions provided herein further include an antioxidant. Exemplary antioxidants include, but are not limited to, ascorbic acid, butylated hydroxyl anisole, butylated hydroxy toluene, propyl gallate, tartaric acid, phosphoric acid, erythrobic acid, lactic acid, sodium sulfite, sodium bisulfate, sodium metabisulfite, thioglycolic acid, cysteine hydrochloride, a tocopherol or combinations thereof. In some embodiments, the antioxidant is at least one compound selected from among ascorbic acid, butylated hydroxyl anisole, butylated hydroxy toluene, propyl gallate, tartaric acid, phosphoric acid, erythrobic acid, lactic acid, sodium sulfite, sodium bisulfate, sodium metabisulfite, thioglycolic acid, cysteine hydrochloride, and tocopherols. The antioxidant may be present in salt forms. In some embodiments, the salt form of an antioxidant includes ascorbic acid, tartaric acid, phosphoric acid, erythrobic acid, and lactic acid. In some embodiments, the antioxidant is a tocopherol. The antioxidant is present in an amount of about 0.025% to about 10%. In one embodiment, the antioxidant is present in an amount of about 0.1% to about 5%.

In some embodiments, a viscosity-modifying polymer is included in the formulation. The viscosity-modifying polymer is selected from the group consisting of a cellulosic polymer, xanthan gum, guar gum, an alginate, acrylates, methacrylates, silicones and polyvinyl pyrrolidone. The viscosity-modifying polymer can be prehydrated in a solvent prior to adding to the final combination of oil and alcohol. The solvent is selected from the group consisting of a water, lower alkyl alcohol, a ketone, a glycol and a mixture thereof. The solvent is at least about 75% by weight of the pre-hydrated viscosity-modifying polymer blend, which can be added at about 0.1-10% by weight to the insecticidal composition to form a topical viscous or gel pharmaceutical formulation.

In some embodiments, the viscosity-modifying polymer includes a cellulosic polymer. In one embodiment, the cellulosic polymer is selected from among hydroxyl ethyl cellulose, hydroxy propyl cellulose, hydroxy propyl methyl cellulose, methyl cellulose, carboxy methyl cellulose, sodium carboxy methyl cellulose, and ethyl cellulose. In some embodiments, the thickener is a ceramide, as described in U.S. Pat. No. 5,665,699.

In some embodiment, the formulation comprises 10-89% essential-oil blend, 5-20% alcohol, 1-20% antioxidant, 0-10% preservative and 5-40% non-essential oil. In another embodiment, the formulation comprises 10-89% essential-oil blend, 5-20% alcohol, 0-20% silicone, 1-20% antioxidant, 0-10% preservative and 5-40% non-essential oil.

Other adjunct minor components may also be incorporated into the compositions provided herein. These ingredients may include coloring agents and opacifiers. Coloring agents and opacifiers include inorganic pigments, e.g., iron oxide, titanium oxide and Prussian Blue, and organic dyestuffs, such as alizarin dyestuffs, azo dyestuffs or metal phthalocyanine dyestuffs, and trace elements, such as salts of iron, manganese, boron, copper, cobalt, molybdenum or zinc may be used. Amounts of these other adjunct minor components may range anywhere from 0.001% up to 20% by weight of the composition.

### C. Preparation of Lippia javanica Essential Oil Compositions

1. Emulsions

In one embodiment, the present invention provides a process of preparing a composition comprising Lippia javanica oil in combination with one or more essential oils as an emulsion. In accordance with the principles of the present invention, the essential-oil combination compositions are prepared as either water or oil based suspensions in a carrier fluid. Known quantities of the essential oils are dispersed into water or oil carrier fluids using agitation or stirring as delivered from machines such as colloid mills, Waring blenders, high-speed homogenizers or lightening mixers. These systems are capable of imparting a large amount of energy into the liquid resulting in the generation of very small drops of one liquid dispersed throughout the other. If water is the continuous phase, it is a water-based suspension. If the continuous phase is oil, it is an oil based suspension. To aid in the dispersion of the essential oils into the carrier fluid, emulsifiers and dispersants may be added. These agents can be non-ionic and/or anionic emulsifying agents (e.g., polyethylene oxide esters of fatty acids, polyethylene oxide ethers of fatty alcohols, alkyl sulfates, alkyl sulfonates, aryl sulfonates, albumin hydrolyzates, and alkyl arylpolyglycol ethers). To stabilize the mixture or to prevent the agglomeration of the droplets over time, the viscosity of the liquid is adjusted using agents such as xanthan gums, polyacryamides or polyacrylates, and swelling clays such as attapulgite, bentonite or veegum. The particle size of the suspended particles may be in the 3 to 5 micron range. Concentrations of the active may range from 0.01 to 70% with the typical concentration approximately 1 to 50% wt/wt.

2. Solutions

In another embodiment, the present invention provides a process of preparing a composition comprising Lippia javanica oil in combination with one or more essential oils in a solution. In one embodiment, the composition includes a fluid carrier that comprises one or more non-essential oils. The method includes liquefying the one or more non-essential oils by heating to 25° C. while stirring with an overhead stirrer, such as a lightning mixer or paddle mixer. The essential oils are blended together and after thorough mixing, are combined with the non-essential oil carrier fluid. In embodiments where antioxidants are included, they are added with the essential-oil blend. In one embodiment, the composition includes essential oils, alcohol and one or more non-essential oils as a carrier fluid, antioxidants, humectants and preservatives. In this embodiment, the method includes liquefying the one or more non-essential oils by heating to 25° C. while stirring; separately combining the essential oils with thorough mixing; combining the non-essential oils and the blend of essential oils with mixing; adding an antioxidant while mixing; mixing any optional skin softening agents, humectants, surfactants or moisturizers to an alcohol solvent and adding a preservative, such as propylparaben, to the alcohol solvent mixture; and combining the oil and alcohol mixtures.

3. Dusts

Dusts are dense powder compositions intended for application in dry form. Dusts are characterized by their free-flowing and rapid settling properties so that they are not readily windborne to areas where their presence is not desired. They contain primarily an active ingredient and a dense, free-flowing, solid extender. Their performance is sometimes aided by the inclusion of a wetting agent.

The dust compositions of this invention comprise from about 0.5 to 40.0 weight percent essential-oil blend and from about 20 to 90 weight percent dense, free-flowing powder. The dusts are made by methods known in the art (e.g., see U.S. Pat. No. 5,413,795, the disclosure of which is incorporated herein by reference.). For example, the free-flowing powder, such as powdered kaolin clays, hydrated attapulgite, or bentonite clays in the form of sodium calcium, or magnesium bentonites, is placed in a surface-coating apparatus such as fluid bed, V-blender or other suitable powder coating apparatus for applying, such as by coating, the essential-oil blend, with or without a solvent, to the carrier powder. A Turbulizer™ apparatus or a Turbulator™ apparatus can be used as the powder coating apparatus. The Turbulizer™ apparatus is manufactured by the Bepex Corporation of Minneapolis, Minn. The use of the Turbulizer™ apparatus is described in more detail in U.S. Pat. No. 5,043,090, the disclosure of which is incorporated herein by reference. The Turbulator™ apparatus is manufactured by Ferro-Tech of Wyandotte, Mich.

4. Granules

Granules are physically stable, particulate compositions containing the essential-oil compositions described herein, which adhere to or are distributed through a basic matrix of a coherent, inert carrier with microscopic dimensions. In order to aid leaching of the active ingredient from the granule, a surfactant may be present. In one embodiment, the inert carrier is of mineral origin, and suitable carriers are natural clays, pyrophyllites or vermiculite. Suitable wetting agents can be anionic or non-ionic.

For the granule compositions of this invention, most suitable carriers are of two types. The first are porous, absorptive pre-formed granules, such as preformed and screened granular attapulgite or heat expanded, granular, screened vermiculite. On either of these, the essential-oil blend can be sprayed and will be absorbed at concentrations up to 25 weight percent of the total weight. The second type are initially powdered kaolin clays, hydrated attapulgite, or bentonite clays in the form of sodium calcium, or magnesium bentonites. Water-soluble salts such as sodium salts may also be present to aid in the disintegrations of the granules in the presence of moisture. These ingredients are blended with the essential-oil blend distributed uniformly throughout the mass. Such granules can also be made with 25 to 30 weight percent essential-oil blend. The granular compositions can be screened to achieve the desired size. In one embodiment, the granule is in a size range of 15-30 mesh.

The most suitable wetting agents for the granular compositions of this invention depend upon the type of granule used. When pre-formed granules are sprayed with essential-oil blend, the most suitable wetting agents are non-ionic, liquid wetters miscible with the solvent. These are more generally known in the art as emulsifiers and comprise alkylaryl polyether alcohols, alkyl polyether alcohols, polyoxethylene sorbitan fatty acid esters, polyethylene glycol esters with fatty or rosin acids, fatty alkylol amide condensates, oil petroleum or vegetable oil sulfonates, or mixtures of these. Such agents will usually comprise up to about 5 weight percent of the total composition. In one embodiment, the granular formulation of this invention comprises about 5 to 30 weight percent essential-oil blend.

5. Aerosols

The compositions herein can be formulated for delivery using a pump spray. In one embodiment, the Lippia javanica essential-oil blend comprises an alcohol, ether or ketone to provide a solution of low viscosity amenable to delivery using a pump spray. In another embodiment, the essential oil composition is diluted 1:1 with ethanol. In another embodiment, the essential oil composition is diluted using 2 parts ethanol to 1 part essential oil composition.

The compositions herein also can be provided in liquefied gases under pressure. Liquefied gaseous extenders or carriers are to be understood as meaning liquids which are gaseous at ambient temperature and under atmospheric pressure, for example aerosol propellants such as butane, propane, nitrogen and carbon dioxide. Aerosol devices are known in the art (e.g., see U.S. Pat. Nos. 3,915,343, 3,884,828, 3,970,584, 4,062,937 and 6,415,992, the disclosure of each of which is incorporated herein by reference in its entirety). Most aerosol products contain the active ingredient and the propellant. The propellant provides the pressure that forces the aerosol product from the container when the valve is opened. Also, the propellant has an influence on whether the product is discharged in the form of a spray, stream, or foam. Variations in the propellant can also influence, for example, whether one obtains a course or fine spray. Examples of suitable propellants include propane, n-butane, isobutane, ethylene, dimethyl ether, nitrogen, nitrous oxide, carbon dioxide and mixtures thereof. No meaningful limitations can be set as to the relative proportions of the individual propellants to be used in combination as this depends on the individual needs and the particular product being formulated.

For example, a method of making an aerosol formulation of the essential oil composition provided herein includes charging the essential oil composition into an aerosol container, fixing the container with an appropriate valve, such as fine-spray, coarse-spray or foam-type valve, vacuum-crimping the valve, filling the aerosol container with the appropriate amount of propellant, and pressurizing the container, such as by introducing nitrogen or nitrous oxide until the desired amount of pressurizing agent is added. As will be appreciated by one skilled in the art, other methods of manufacture such as in-line blending of essential-oil blend and propellants or sparging the nitrous oxide may be used.

In one embodiment, the essential-oil blend provided herein is mixed with a portion of a propellant to form a homogeneous concentrate mixture. The concentrate is then placed in an aerosol container, the aerosol container is sealed with a suitable aerosol valve, and the aerosol container is pressurized with the remainder of propellant. Finally, the aerosol container is shaken and a stable aerosol suspension is thereby obtained.

### D. Formulations

Formulations containing the compositions of the present invention may be prepared in any known manner, for instance by extending the compositions with conventional pesticide dispersible liquid carriers and/or dispersible solid carriers. Suitable liquid diluents or carriers include water, petroleum distillates, or other liquid carriers with or without surface-active agents. The choice of dispersing and emulsifying agents and the amount employed is dictated by the nature of the composition and the ability of the agent to facilitate the dispersion of the compositions provided herein. The compositions provided herein also may be formulated for topical administration to a subject, or for surface administration to a locus to be treated.

Topical Formulations

Topical mixtures are prepared as described for local administration. The resulting mixture can be a solution, suspension or emulsion and the compositions may be formulated as creams, gels, ointments, emulsions, solutions, elixirs, lotions, suspensions, tinctures, pastes, foams, aerosols and sprays, or any other formulations suitable for topical administration.

The compounds may be formulated for local or topical application, such as for topical application to the skin or hair, in the form of gels, creams, lotions, oils, shampoo, conditioner, rinses, liquids, hair sprays, mousse, etc. using methods known to the skilled artisan (e.g., see Flick, “Cosmetic and Toiletry Formulations Volume 8 (Cosmetic & Toiletry Formulations),” Noyes Publications (2001); Cosmetic Formulation of Skin Care Products (Cosmetic Science and Technology Series Vol. 30), Draelos and Thaman, eds., Informa Healthcare (1st edition, 2006); Schlossman, “The Chemistry and Manufacture of Cosmetics: Formulating (Chemistry and Manufacture of Cosmetics),” Allured Publishing Corporation (3rd edition, 2002); U.S. Pat. No. 5,472,686; Davis, Aromatherapy—An A-Z, Revised and Enlarged Edition (1995), pages 354-358; Rose, The Aromatherapy Book—Applications & Inhalations, North Atlantic Books (1992)).

In certain embodiments, the composition is prepared for topical administration. Certain of such compositions contain bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for the topical composition provided herein can be chosen in view of the particular ectoparasite or pest to be repelled or eradicated. In certain embodiments, the composition is administered as a single application. In certain embodiments, a composition is administered as a series of two or more applications administered over one or more days. One skilled in the art can determine an effective amount of the composition of the invention for including in a topical formulation for killing or repelling ectoparasites and pests.

### E. Methods of Use of the Compositions

It will be appreciated by the skilled artisan that the pesticidal compositions disclosed herein exhibit excellent pesticidal activities at short exposure times, such as less than 30 minutes, less than 10 minutes, less than 5 minutes or less than 2 minutes.

Use of pesticidal compositions of the present invention generally results in 100% knockdown on contact or within 10 minutes of contact. Use of pesticidal compositions of the present invention generally results in 100% mortality. As such, the compositions provided herein are advantageously employed as pesticidal agents in uses such as, without limitation, agriculture, households, professional pest control, and pet bedding, and against mosquitoes, flies, fleas, ants, lice including head lice, dust mites, and other ectoparasites and pests.

Provided herein are methods for killing, combating or controlling ectoparasites or pests. In one embodiment, the method comprises applying to at least one of (a) such ectoparasites or pests and (b) the locus to be protected, e.g., an area in which the ectoparasite or pest is to be repelled or eradicated, such as a bedding area, a room, a house, clothing, upholstery or to a subject, such as a mammal including a human or an animal, such as a domestic animal, a pesticidally effective amount, or toxic amount, or an amount effective for repelling an ectoparasite or pest, of a composition provided herein, alone or together with a carrier as noted above. The formulations or compositions provided herein may be applied in any suitable usual manner, for instance by spraying, atomizing, vaporizing, scattering, dusting, watering, squirting, sprinkling, or pouring. The method for controlling insects comprises applying a composition provided herein, ordinarily in a formulation of one of the aforementioned types, to a locus or area to be protected from the insects, such as the bedding area or living area of a subject or to an environment in which the ectoparasite or pest lives, such as carpeting, bedding, fabric, upholstery or clothing. The composition is applied in an amount sufficient to effect the desired action. This dosage is dependent upon many factors, including the targeted ectoparasite or pest, the carrier employed, the method and conditions of the application, whether the formulation is present at the locus in the form of an aerosol, or as a film, or as discrete particles, and the thickness of film or size of particles. Proper consideration and resolution of these factors to provide the necessary dosage of the active compound at the locus to be protected are within the skill of those versed in the art. In general, however, the effective dosage of the compound of this invention at the locus to be protected, i.e., the dosage with which the pest comes in contact, is of the order of 0.001 to 90% based on the total weight of the formulation being applied. In some embodiment, the effective concentration will be as little as 0.0001% to 10%. In other embodiments, the effective concentration will be as much as 50% to 90%.

In another embodiment, provided herein is a method for treating an ectoparasite on a subject, comprising the steps of topically applying to the subject a therapeutically effective amount of a composition provided herein, where the composition comprises: a) at least 3% Lippia javanica oil in combination with one or more essential oils; and b) a carrier.

In another embodiment, provided herein is a method for treating an ectoparasite on a subject, comprising the steps of topically applying to the subject a therapeutically effective amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for treating an ectoparasite on a subject, comprising the steps of topically applying to the subject a therapeutically effective amount of a composition provided herein, wherein the composition comprises: a) at least 5% Lippia javanica oil in combination with one or more essential oils; b) a non-essential oil; and c) an alcohol, wherein the composition is applied in the form of an oil, a lotion, a cream or a foam.

In one embodiment, the composition provided herein is applied to the subject for a time sufficient to kill the ectoparasites. In one embodiment, the composition is applied for about 30 minutes. In one embodiment, the composition is applied for about 20 minutes. In one embodiment, the composition is applied for about 10 minutes. In one embodiment, the composition is applied for about 5 minutes.

In one embodiment, the method includes application of a composition provided herein to the hair or skin of the subject so that the hair or the skin is totally saturated with the composition. In one embodiment, the composition is applied more than once, if required. In another embodiment, the composition is applied periodically as a preventative. In one embodiment, the composition is applied every two weeks. In another embodiment, the composition is applied monthly.

In one embodiment, a composition provided herein is to be applied to a subject in an amount sufficient to effect the desired action. The dosage applied is dependent upon many factors, including the targeted ectoparasite or pest, the carrier employed, the area of the subject to which the application is to be applied, such as the hair or the skin, the method and conditions of the application, whether the formulation is applied in the form of an aerosol, or as a film, such as in an oil or lotion, and the thickness of the film resulting from the method of application. Proper consideration and resolution of these factors to provide the necessary dosage of the active compound at the locus of application on the subject are within the skill of those versed in the art. In one embodiment, a composition provided herein is applied in an amount applied effective to totally saturate the scalp hair, the body hair and/or the skin of affected area of the body of the subject. In one embodiment, the composition is applied to the hair/the body in the amount of about 50 grams. By totally saturating the hair or the body, all of the hair, from the root to the tips, will be covered and saturated with the insecticide composition provided herein, thereby making sure that all ectoparasites are exposed to the composition provided herein.

The compositions provided herein are applied for a shorter duration of time (e.g., 5 minutes to 60 minutes) as compared to existing commercial products instructed to have application time of 8-12 hours or overnight. In one embodiment, the composition is applied to the target area, such as an area of infestation, and allowed to remain in contact with the hair and/or skin of the target area for a duration of between about 5 minutes to about 30 minutes. In another embodiment, the composition is applied to the target area, such as an area of infestation, and allowed to remain in contact with the hair and/or skin of the target area for a duration of between about 10 minutes to about 20 minutes. In another embodiment, the composition is applied to the target area and allowed to remain in contact with the hair and/or skin of the target area for a duration of less than 15 minutes. In another embodiment, the composition is applied to the target area and allowed to remain in contact with the hair and/or skin of the target area for a duration of less than 10 minutes. In another embodiment, the composition is applied to the target area and allowed to remain in contact with the hair and/or skin of the target area for a duration of 5 minutes or less than 5 minutes.

The pesticidal compositions and methods provided herein are effective against a wide variety of ectoparasites and pests, and it will be understood that the ectoparasites and pests exemplified and evaluated in the working examples herein are representative of such a wider variety or ectoparasites and pests. For instance, pesticidal compositions and methods provided herein are effective to kill or repel insects (Insecta), true bugs (Hemiptera), flies (Diptera), fleas (Siphonaptera), spiders (Arachnida), ticks and mites (Acari), lice (Phthiraptera), ants, no-see-ums, wall lice, horse lice, chinch bugs, redcoats, bedbugs, chiggers, drain flies, weevils, aphids, white flies, ants, millipedes, centipedes, carpet beetles, spiders, gnats, fruit flies, cluster flies, phorid flies and black flies.

The pesticidal compositions and methods provided herein are effective in repelling or killing ectoparasites or pests on a wide variety of subjects. For instance, the present invention may be used to control pests that attack warm-blooded animals. Representative animals that may be protected or treated by the present invention include, without limitation, humans, horses, dogs, cats, cattle, sheep, goats, hogs, rabbits, guinea pigs, hamsters, mice, rats and gerbils.

In another embodiment, provided herein is a method for killing ectoparasites or pests, comprising the steps of topically applying to the ectoparasites or pests an amount of a composition provided herein effective to kill the ectoparasites or pests, wherein the composition comprises at least 5% Lippia javanica oil in combination with one or more essential oils. In another embodiment, provided herein is a method for killing ectoparasites or pests, comprising the steps of topically applying to the ectoparasites or pests an amount of a composition provided herein effective to kill the ectoparasites or pests, wherein the composition comprises at least 5% Lippia javanica oil in combination with one or more essential oils, wherein the ectoparasites or pests are selected from among insects (Insecta), true bugs (Hemiptera), flies (Diptera), fleas (Siphonaptera), spiders (Arachnida), ticks and mites (Acari), lice (Phthiraptera).

In another embodiment, provided herein is a method for killing ectoparasites or pests, comprising the steps of topically applying to the ectoparasites or pests an amount of a composition provided herein effective to kill the ectoparasites or pests, wherein the composition comprises at least 5% Lippia javanica oil in combination with one or more essential oils, wherein the ectoparasites or pests are selected from among head lice, body lice, pubic lice, chiggers, scabies, flies, midges, mosquitoes, no-see-ums, wall lice, horse lice, chinch bugs, redcoats, bedbugs, chiggers, fleas (including sand fleas, snow fleas and human fleas), drain flies, mites, weevils, aphids, white flies, ants, gnats, millipedes, centipedes, carpet beetles, spiders, fruit flies, cluster flies, phorid flies and black flies.

The compositions provided herein also are used to combat ectoparasite infestation by treating articles of clothing, including hats, caps, shower caps, helmet liners and headbands, and for treating bedding, sheets, pillows, blankets, and for treating personal care products including brushes and combs.

1. Lice

The number of cases of humans infested with lice has increased worldwide, reaching hundreds of millions yearly (e.g., see Taplin et al., Semin Dermatol 6: 125-135 (1987)). It is estimated that, in the U.S., the number of people treated for head lice infestation is between about 6 to 12 million people, many of whom are children (Williams et al., Pediatrics 107(5): 1011-1015 (2001)). Some school districts in the U.S. have implemented “no nit” policies, which exclude children who have any lice nits from attending school, resulting in a loss of about 12-24 million school days in 1998 (e.g., see Price et al., J Sch Health 69: 153-158 (1999)).

Lice are the most common ectoparasites, and include head lice, body lice, pubic lice and scabies. Head lice (Pediculus humanus capitis) is a persistent health problem. For example, several million school children in United States will become infected with head lice annually. Head lice infestation is produced by the common head louse Pediculus humanus capitis, and typically causes itching of the scalp. As the lice feed on human blood, they may cause lesions to develop on the scalp, swollen glands on the neck or underarms, or other symptoms. Head lice infestation causes serious problems due to the negative social implications of the infestation. Body lice are also bothersome to humans and carry the additional hazard of being the vectors of certain diseases, such as exanthematic or epidemic typhus and recurrent fever. Evidence also suggests that head lice may be vectors for systemic human infections (see, e.g., U.S. Pat. No. 6,103,248).

The pharmaceutical Ovide® Lotion is used to kill head lice. Ovide® Lotion contains malathion and a high percentage of isopropyl alcohol. The patient is instructed to apply the lotion for 8-12 hours.

Other treatments against ectoparasites such as lice also exist. Several products for treating pediculosis capitis are available over the counter. These include Rid® Lice Killing Shampoo (Pfizer), Nix® Cream Rinse and A-2000® Shampoo Concentrate, which contains pyrethins and piperonyl butoxide as active ingredients. Another prescription treatment is Kwell® (containing lindane 1%). Also available are permethrin cream (5%), pyrethrin shampoo, crotamiton cream, or oral dosage of ivermectin. Permethrin and lindane treatment requires applying the drugs and allowing them to remain on the skin and/or hair for extended periods of time. The manufacturers claim appropriate use of these products does not lead to resistance but evidence now would seem to suggest otherwise. Studies have shown lice are resistant to many of these treatments. In light of this, some health authorities are now advising no active treatment using these compounds because of problems apparently due to resistant lice.

Many of these drugs are either toxic to the nervous system or cause allergic reactions. For example, lindane cannot be used on infants, children, pregnant/nursing women, or people that suffer from seizures or other neurological diseases. Lindane is banned in the state of California and in 52 countries.

The compositions provided herein are effective in killing lice when applied to a subject. The time to kill the ectoparasites is significantly reduced when compared to traditional treatment regimes. The method comprises applying to the hair, scalp and/or skin of a subject in need of treatment for lice an amount of a composition provided herein sufficient to kill the lice. In one embodiment, the composition is effective to kill head lice, body lice, pubic lice or to treat scabies.

In another embodiment, provided herein is a method to kill head lice and nits, comprising applying to a subject thusly infested, an amount effective to kill lice and nits, of an essential-oil blend provided herein. In one embodiment, the essential-oil blend is in an acceptable carrier for topical application. In one embodiment, the essential-oil blend comprises at least 3% Lippia javanica oil in combination with one or more essential oils. In another embodiment, the method comprises applying a composition comprising 3% Lippia javanica oil in combination with one or more essential oils selected from among eucalyptus oil, lemongrass oil, petitgrain oil, Rosmarinum officinalis (rosemary) oil, Thymus vulgaris (thyme) oil, Lavendula augustifolia (lavender) oil, Melaleuca alternifolia (tea tree) oil, Tagete minuta oil, Levisticum officinalis (lovage) oil, cinnamon oil, lemon oil and oil of bergamot.

In another embodiment, the method comprises applying a composition comprising Lippia javanica oil and Lavendula augustifolia oil. In one embodiment, the method comprises applying a composition comprising Lippia javanica oil and Lavendula augustifolia oil in a ratio of 3:2. In another embodiment, the method comprises applying a composition comprising Lippia javanica oil and Lavendula augustifolia oil in combination with one or more essential oils selected from among eucalyptus oil, lemongrass oil, petitgrain oil, Rosmarinum officinalis oil, Thymus vulgaris oil, Melaleuca alternifolia oil, Tagete minuta oil, Levisticum officinalis oil, and lemon oil.

In another embodiment, the method comprises applying a composition comprising Lippia javanica oil and eucalyptus oil. In another embodiment, the method comprises applying a composition comprising Lippia javanica oil and eucalyptus oil in combination with one or more essential oils selected from among lemongrass oil, petitgrain oil, Rosmarinum officinalis oil, Thymus vulgaris oil, Lavendula augustifolia oil, Melaleuca alternifolia oil, Tagete minuta oil, Levisticum officinalis oil, lemon oil and oil of bergamot.

In another embodiment, the method comprises applying a composition comprising Lippia javanica oil and lemongrass oil. In one embodiment, the method comprises applying a composition comprising Lippia javanica oil and lemongrass oil in a ratio of 1:1. In another embodiment, the method comprises applying a composition comprising Lippia javanica oil and lemongrass oil in combination with one or more essential oils selected from among eucalyptus oil, petitgrain oil, Rosmarinum officinalis oil, Thymus vulgaris oil, Lavendula augustifolia oil, Melaleuca alternifolia oil, Tagete minuta oil, Levisticum officinalis oil, cinnamon oil, lemon oil and oil of bergamot.

In another embodiment, the method comprises applying a composition comprising Lippia javanica oil and Rosmarinum officinalis oil. In one embodiment, the method comprises applying a composition comprising Lippia javanica oil and Rosmarinum officinalis oil in a ratio of 3:1. In another embodiment, the method comprises applying a composition comprising Lippia javanica oil and Rosmarinum officinalis oil in combination with one or more essential oils selected from among eucalyptus oil, lemongrass oil, petitgrain oil, Thymus vulgaris (thyme) oil, Lavendula augustifolia (lavender) oil, Melaleuca alternifolia (tea tree) oil, Tagete minuta oil, Levisticum officinalis (lovage) oil, cinnamon oil, lemon oil and oil of bergamot.

In another embodiment, the method comprises applying a composition comprising Lippia javanica oil and Melaleuca alternifolia oil. In one embodiment, the method comprises applying a composition comprising Lippia javanica oil and Melaleuca alternifolia oil in a ratio of from about 3:4 to about 4:3. In another embodiment, the method comprises applying a composition comprising Lippia javanica oil and Melaleuca alternifolia oil in combination with one or more essential oils selected from among eucalyptus oil, lemongrass oil, petitgrain oil, Rosmarinum officinalis (rosemary) oil, Thymus vulgaris (thyme) oil, Lavendula augustifolia (lavender) oil, Melaleuca alternifolia (tea tree) oil, Tagete minuta oil, Levisticum officinalis (lovage) oil, cinnamon oil, lemon oil and oil of bergamot.

In another embodiment, the method comprises applying a composition comprising Lippia javanica oil, Rosmarinum officinalis oil and Thymus vulgaris oil. In another embodiment, the composition comprises Lippia javanica oil, Rosmarinum officinalis oil and Thymus vulgaris oil in combination with one or more essential oils selected from among eucalyptus oil, lemongrass oil, petitgrain oil, Lavendula augustifolia oil, Melaleuca alternifolia oil, Tagete minuta oil, Levisticum officinalis oil, cinnamon oil, lemon oil and oil of bergamot.

In another embodiment, the method comprises applying a composition comprising Lippia javanica oil, Rosmarinum officinalis oil, Thymus vulgaris oil and Lavendula augustifolia oil. In another embodiment, the composition comprises Lippia javanica oil, Rosmarinum officinalis oil, Thymus vulgaris and Lavendula augustifolia oil in combination with one or more essential oils selected from among eucalyptus oil, lemongrass oil, petitgrain oil, Melaleuca alternifolia oil, Tagete minuta oil, Levisticum officinalis oil, cinnamon oil, lemon oil and oil of bergamot.

In another embodiment, the method comprises applying a composition comprising Lippia javanica oil, Rosmarinum officinalis oil, Thymus vulgaris oil and Melaleuca alternifolia oil. In another embodiment, the method comprises applying a composition comprising Lippia javanica oil, Rosmarinum officinalis oil, Thymus vulgaris and Melaleuca alternifolia oil in combination with one or more essential oils selected from among eucalyptus oil, lemongrass oil, petitgrain oil, Lavendula augustifolia (lavender) oil, Tagete minuta oil, Levisticum officinalis (lovage) oil, cinnamon oil, lemon oil and oil of bergamot.

In another embodiment, the method comprises applying a composition comprising Lippia javanica oil, lemongrass oil, Rosmarinum officinalis oil, Thymus vulgaris oil and Melaleuca alternifolia oil. In another embodiment, the method comprises applying a composition comprising Lippia javanica oil, lemongrass oil, Rosmarinum officinalis oil, Thymus vulgaris and Melaleuca alternifolia oil in combination with one or more essential oils selected from among eucalyptus oil, petitgrain oil, Lavendula augustifolia (lavender) oil, Tagete minuta oil, Levisticum officinalis (lovage) oil, cinnamon oil, lemon oil and oil of bergamot.

In another embodiment, the method comprises applying a composition comprising Lippia javanica oil, lemongrass oil, Lavendula augustifolia oil, Rosmarinum officinalis oil, Thymus vulgaris oil and Melaleuca alternifolia oil. In another embodiment, the method comprises applying a composition comprising Lippia javanica oil, lemongrass oil, Lavendula augustifolia oil, Rosmarinum officinalis oil, Thymus vulgaris and Melaleuca alternifolia oil in combination with one or more essential oils selected from among eucalyptus oil, petitgrain oil, Tagete minuta oil, Levisticum officinalis (lovage) oil, cinnamon oil, lemon oil and oil of bergamot.

In one embodiment, the method comprises applying a composition comprising Lippia javanica oil and eucalyptus oil. In another embodiment, the method comprises applying a composition comprising Lippia javanica oil, lemongrass oil and lemon oil. In another embodiment, the method comprises applying a composition comprising Lippia javanica oil, lavender oil, rosemary oil, and petitgrain oil. In another embodiment, the method comprises applying a composition comprising Lippia javanica oil, tea tree oil, lavender oil and eucalyptus oil. In another embodiment, the method comprises applying a composition comprising Lippia javanica oil, Tagete minuta oil and cinnamon oil. In another embodiment, the method comprises applying a composition comprising Lippia javanica oil, Tagete minuta oil and Levisticum officinalis oil.

In another embodiment, the method comprises (a) topically applying a composition provided herein to the infestation; and (b) allowing the composition to remain on the infestation for at least about ten minutes. In another embodiment, the method comprises (a) topically applying a composition provided herein to the infestation; and (b) allowing the composition to remain on the infestation for at least about five minutes.

In another embodiment, provided herein is a method of treating an infestation of nits of head lice, in both mature and immature forms, comprising: (a) topically applying a composition provided herein to the infestation; and (b) allowing the composition to remain on the infestation for at least about ten minutes. In another embodiment, provided herein is a method of treating an infestation of nits of head lice, in both mature and immature forms, comprising: (a) topically applying a composition provided herein to the infestation; and (b) allowing the composition to remain on the infestation for at least about five minutes.

In another embodiment, provided herein is a method of treating a head lice infestation, in both mature and immature forms, comprising: (a) topically applying a composition provided herein to the infestation; and (b) allowing the composition to remain on the infestation for at least about five minutes. In one embodiment, the method further includes the step of combing the hair in the affected area a section at a time after treatment to remove lice and nits. In another embodiment, the method further includes the step of shampooing the hair after combing.

In another embodiment, provided herein is a method to repel head lice, comprising applying topically to a subject susceptible to lice infestation an amount of a composition provided herein effective to repel but not kill lice.

In another embodiment, provided herein is a method to repel lice, including head lice. The method comprises adding a combination of essential oils as provided herein in an amount effective to repel head lice to a shampoo, a conditioner, a hair spray, a hair rinse, a styling gel or other haircare product, and applying the haircare product to the hair of a subject, whereby lice are repelled. The repellent compositions also are effective as a repellent when applied to clothing, upholstery, furniture, carpeting and bedding.

The head lice repellent and treatment for killing head lice may be prepared in various forms, such as an aqueous emulsion, an alcohol solution, a cream, a powder, a gel, a rinse, a spray, hair styling mousse, shampoo, or conditioner.

The head lice repellent and treatment for killing head lice may be delivered in various forms, such as a pump, a pump spray, a spray mist, an aerosol spray, a rinse, a liquid, a dip for brushes, combs, and a liquid, spray, foam or powder for treating material, such as bedding material and/or fabric or for application to a subject. Also provided is a method of preventing lice infestation, comprising applying topically to a subject susceptible to lice infestation an amount of a composition provided herein effective to repel lice. The application of the composition to the subject may be repeated, such as once a week, every other week or once a month in order to prevent infestation with head lice.

In another embodiment, a method of killing lice and/or nits is provided, the method comprising washing the clothing or bedding materials infested with lice in hot water, and adding the essential oil composition provided herein to the wash cycle or rinse cycle or both, thereby killing the lice and/or nits. In one embodiment, at least 1 ounce of the essential oil compositions provided herein is added to the wash or rinse cycle.

2. Fleas

Fleas are known disease carriers for both the bubonic and pneumonic plagues. The spreading of a plague by fleas transmitting disease from animals such as rodents to human beings has much potential in the United States. It is particularly problematic in third-world countries where the standard of living is low and living conditions are conducive for transmitting such disease from animals to human beings. Therefore, removing fleas from domestic animals is more than just removing an irritation.

Fleas, which belong to the insect order Siphonaptera, are obligate ectoparasites for a wide variety of animals, including birds and mammals. Flea infestation of animals is of health and economic concern because fleas are known to cause and/or transmit a variety of diseases. Fleas cause and/or carry infectious agents that cause, for example, flea allergy dermatitis, anemia, murine typhus, plague and tapeworm. In addition, fleas are a problem for animals maintained as pets because the infestation becomes a source of annoyance for the pet owner who may find his or her home generally contaminated with fleas which feed on the pets. As such, fleas are a problem not only when they are on an animal but also when they are in the general environment of the animal.

Flea infestation is commonly treated with insecticides in formulations such as sprays, shampoos, dusts, dips, or foams, or in pet collars. While some of these products are efficacious, most, at best, offer protection of a very limited duration. Furthermore, many of the methods are often not successful in reducing flea populations on the pet.

Provided herein are methods for killing fleas on a subject. In one embodiment, provided herein is a method for killing fleas on a subject, comprising the steps of topically applying to the subject an amount of a composition provided herein effective to kill fleas, where the composition comprises: a) at least 3% Lippia javanica oil in combination with one or more essential oils; and b) a carrier.

In another embodiment, provided herein is a method for killing fleas on a subject, comprising the steps of topically applying to the subject a therapeutically effective amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for killing fleas on a subject, comprising the steps of topically applying to the subject a therapeutically effective amount of a composition provided herein, wherein the composition comprises: a) at least 5% Lippia javanica oil in combination with one or more essential oils; b) a non-essential oil; and c) an alcohol, wherein the composition is applied in the form of an oil, a lotion, a cream or a foam.

In one embodiment, a composition provided herein is applied to the subject for a time sufficient to kill the fleas. In one embodiment, the composition is applied for about 30 minutes. In one embodiment, the composition is applied for about 20 minutes. In one embodiment, the composition is applied for about 10 minutes. In one embodiment, the composition is applied for about 5 minutes.

In another embodiment, provided herein is a method of killing fleas in a locus where such flea control is desired. In one embodiment, provided herein is a method for killing fleas, comprising the steps of topically applying to the fleas or the environment or locus exhibiting flea infestation an amount of a composition provided herein effective to kill fleas, where the composition comprises: a) at least 1% Lippia javanica oil in combination with one or more essential oils; and b) a carrier.

In another embodiment, provided herein is a method for killing fleas, comprising the steps of topically applying to the flea or the locus infested therewith a therapeutically effective amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided is a method of killing fleas in a carpet, comprising dispersing or mixing a composition provided herein with one or more dispersing powders selected from among an inorganic sulfate, bicarbonate or chloride, an aluminum silicate clay to form a powdered dispersion of the composition, applying the powdered dispersion of the composition uniformly to the carpet and thereafter removing it from the carpet. Methods of making a carpet powder are known in the art (for example, see U.S. Pat. No. 4,873,000). In addition to powders, in one embodiment provided herein is a foam composition for application to a carpet for killing fleas. The composition includes the essential-oil blend disclosed herein formulated in a friable foam carpet formulation, such as the formulation described in U.S. Pat. No. 6,315,949.

In another embodiment, provided herein is a method of killing fleas in a vacuum cleaner, comprising dispersing or mixing a composition provided herein with rock salt or a granule, such as granular attapulgite or heat expanded, granular, screened vermiculite to form a granular dispersion of the composition, and placing the granular dispersion of the composition into the dust collecting bag of a vacuum.

In another embodiment, provided herein is a method to repel fleas, comprising applying topically to a subject susceptible to flea infestation an amount of a composition provided herein effective to repel but not kill fleas.

In another embodiment, the method comprises adding a combination of essential oils as provided herein in an amount effective to repel fleas to a shampoo, a conditioner, a hair spray, a hair rinse, a styling gel or other haircare product, and applying the haircare product to the hair of a subject, whereby fleas are repelled. The repellent compositions also are effective as a repellent when applied to clothing, furniture, upholstery, carpeting and bedding.

The flea repellent compositions provided herein also are absorbed into or adsorbed onto a receptive or porous material, such as a pliable plastic, that can be formed into a loop, e.g., as in a flea collar. In one embodiment, provided herein is a method for repelling fleas that comprises absorbing into or adsorbed onto a receptive or porous material a combination of essential oils as provided herein in an amount effective to repel fleas to form a flea repelling article, and placing the flea repelling article on the subject to repel fleas.

The flea repellent and treatment for killing fleas may be prepared in various forms, such as an aqueous emulsion, an alcohol solution, a cream, a powder, a gel, a rinse, an oil, a foam, a spray, a hair styling mousse, a shampoo, or conditioner.

The flea repellent and treatment for killing fleas may be delivered in various forms, such as a pump, a pump spray, a spray mist, an aerosol spray, a rinse, a liquid, a dip for brushes, combs, and liquids, sprays or foams for treating material, such as bedding material and/or fabric or for application to a subject. The flea repellent and treatment for killing fleas is applied once and may be repeated at intervals including weekly, every two weeks, once a month or every other month.

In another embodiment, a method of killing fleas is provided, the method comprising washing clothing, upholstery or bedding materials infested with fleas in hot water, and adding an essential-oil composition provided herein to the wash cycle or rinse cycle or both, thereby killing the fleas. In one embodiment, at least 1 ounce of an essential-oil composition provided herein is added to the wash or rinse cycle.

3. Mosquitoes

The mosquito is a member of the family Culicidae. Mosquitoes are now considered the most dangerous insects in the world, killing an estimated two to three million people per year by transmitting disease, such as malaria and West Nile virus, through their bites. There are hundreds of different species of mosquito worldwide. For example, there are more than 150 species in the United States. The most common species found in the U.S. include the Aedes albopictus, Culex pipiens and Anopheles quadrimaculatus. In most instances, each species carries different types of diseases and will typically breed and feed at different times of the day. Aedes albopictus is also known as the Asian tiger mosquito and is associated with the transmission of dengue fever, eastern equine encephalitis and heartworm. It generally feeds during the daytime. Culex pipiens is also known as the northern house mosquito. It is the most common species found in urban areas. This species of mosquito is associated with the transmission of encephalitis and the transmission of the West Nile virus to humans, birds and other mammals. It generally feeds at dusk. Anopheles quadrimaculatus is the mosquito associated with the transmission of malaria in the eastern, central and southern U.S. It generally feeds after dusk and just before dawn.

At the present time, N,N-diethyl-m-toluamide (DEET) is the active ingredient included in most commercial topical insect repellents. It is used to repel biting pests such as mosquitoes and ticks. It is estimated that nearly one-third of the U.S. population uses DEET annually. Based on extensive toxicity testing, the EPA believes that the normal use of DEET does not present a health concern to the general population. Dermal application of insect repellents containing DEET can produce a variety of skin reactions in humans. Cases of localized skin irritation, large painful blisters and permanent scarring of skin at the crease of the elbow have been reported. DEET is a synthetic chemical having a noticeably unpleasant odor, which results in low user acceptability. Formulations with greater than 10% DEET should not be used on children.

There have been efforts to develop a natural insect repellent. For example, catnip (Nepeta cataria), Osage orange (Maclura pomiferia), Amyris (Amyris balsamifera) and siam wood (Fokienia hodginsii) essential oils are reported to have repellent properties to several insect species (Schultz et al., “Mosquito repellency to plant essential oil mixtures,” Entomological Soc. of America Annual Meeting, 2006).

Provided herein are methods for killing mosquitoes. In one embodiment, provided herein is a method for killing mosquitoes, comprising the steps of topically applying to the mosquitoes an amount of a composition provided herein effective to kill mosquitoes, where the composition comprises: a) at least 3% Lippia javanica oil in combination with one or more essential oils; and b) a carrier.

In another embodiment, provided herein is a method for killing mosquitoes, comprising the steps of topically applying to the mosquitoes a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for killing mosquitoes, comprising the steps of topically applying to the mosquitoes a killing amount of a composition provided herein, wherein the composition comprises: a) at least 5% Lippia javanica oil in combination with one or more essential oils; b) a non-essential oil; and c) an alcohol, wherein the composition is applied in the form of an oil, a lotion, a cream or a foam.

In another embodiment, provided herein is a method of killing larvae of mosquitoes in a locus where such mosquito control is desired. In one embodiment, provided herein is a method for killing larvae of mosquitoes, comprising the steps of applying to the larvae of mosquitoes or to the surface of water exhibiting mosquito larvae infestation an amount of a composition provided herein effective to larvae of mosquitoes, where the composition comprises: a) at least 3% Lippia javanica oil in combination with one or more essential oils; and b) a carrier.

In another embodiment, provided herein is a method for killing larvae of mosquitoes, comprising the steps of applying to the larvae of mosquitoes or to the surface of water exhibiting mosquito larvae infestation an amount of a composition provided herein effective to kill larvae of mosquitoes, where the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for killing mosquitoes, comprising the steps of dispersing an amount of a composition provided herein into the collector of an apparatus for attracting and collecting insects, such as described in U.S. Pat. No. 6,594,944, and attracting the mosquitoes to the apparatus by producing carbon dioxide and/or heat and/or moisture, whereby the mosquitoes are captured in the collector, which topically applies the composition disclosed herein to the mosquito, killing the mosquito. In one embodiment, the method includes topically applying to the mosquitoes a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method to repel mosquitoes, comprising applying topically to a subject susceptible to mosquito bites an amount of a composition provided herein effective to repel but not kill mosquitoes.

In another embodiment, the method comprises adding a combination of essential oils as provided herein in an amount effective to repel mosquitoes to a shampoo, a conditioner, a hair spray, a hair rinse, a styling gel or other haircare product, or lotion, cream, gel or other skincare product and applying the haircare product to the hair or the skincare product to the skin of a subject, whereby mosquitoes are repelled. The repellent compositions also are effective as a repellent when applied to clothing, furniture, upholstery, carpeting and bedding.

The mosquito repellent compositions provided herein also may be absorbed into or adsorbed onto a receptive or porous material, such as a pliable plastic. In one embodiment, provided herein is a method for repelling mosquitoes that comprises absorbing into or adsorbed onto a receptive or porous material a composition comprising at least 5% Lippia javanica essential oil in combination with one or more essential oils as provided herein in an amount effective to repel mosquitoes to form a mosquito repelling article, and placing the mosquito repelling article in an environment in which abatement of mosquitoes is desired, wherein the essential-oil composition provided herein is transferred from the mosquito repelling article into the environment, thereby repelling mosquitoes.

The mosquito repellent and treatment for killing mosquitoes may be prepared in various forms, such as an aqueous emulsion, an alcohol solution, a cream, a lotion, a powder, a gel, a rinse, a spray, an oil, a hair styling mousse, a shampoo, or a conditioner.

The mosquito repellent and treatment for killing mosquitoes may be delivered in various forms, such as a pump, a pump spray, a spray mist, an aerosol spray, a rinse, or liquid, and for treating material, such as bedding material and/or fabric or for application to a subject.

4. Flies

Flies are common pests that are not only a nuisance but also a vector for diseases. The most common fly pests include the premise flies, such as the common housefly and stable flies, and the pasture flies, which include horn flies, horse flies, face flies and heel flies. The cattle industry in the U.S. alone loses millions of dollars annually due to ectoparasites including lice, flies and ticks (Wright et al., “Beef Cattle Ectoparasites,” Oklahoma Cooperative Extension Fact Sheet F-7000). Other nuisance flies include, but are not limited to, midges, gnats, no-see-ums, drain flies, white flies, and black flies.

Provided herein are methods for killing flies. In one embodiment, provided herein is a method for killing flies, comprising the steps of topically applying to the flies an amount of a composition comprising at least 5% Lippia javanica essential oil in combination with one or more essential oils provided herein effective to kill flies, where the composition comprises: a) at least 5% Lippia javanica oil in combination with one or more essential oils; and b) a carrier.

In another embodiment, provided herein is a method for killing flies, comprising the steps of topically applying to the flies a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method of killing larvae of flies in a locus where such fly control is desired. In one embodiment, provided herein is a method for killing larvae of flies, comprising the steps of applying to the larvae of flies an amount of a composition provided herein effective to kill larvae of flies, where the composition comprises: a) at least 3% Lippia javanica oil in combination with one or more essential oils; and b) a carrier.

In another embodiment, provided herein is a method for killing larvae of flies, comprising the steps of applying to the larvae of flies an amount of a composition provided herein effective to larvae of flies, where the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method to repel flies, comprising applying topically to a subject an amount of a composition comprising at least 3% Lippia javanica oil in combination with one or more essential oils provided herein effective to repel but not kill flies.

In another embodiment, the method comprises adding a composition comprising at least 3% Lippia javanica oil in combination with one or more essential oils as provided herein in an amount effective to repel flies to a shampoo, a conditioner, a hair spray, a hair rinse, a styling gel or other haircare product, or lotion, cream, gel or other skincare product and applying the haircare product to the hair or the skincare product to the skin of a subject, whereby flies are repelled. The repellent compositions also are effective as a repellent when applied to clothing, furniture, and bedding.

The fly repellent compositions provided herein may also be absorbed into or adsorbed onto a receptive or porous material, such as a pliable plastic. In one embodiment, provided herein is a method for repelling flies that comprises absorbing into or adsorbed onto a receptive or porous material a composition comprising at least 3% Lippia javanica oil in combination with one or more essential oils as provided herein in an amount effective to repel flies to form a fly repelling article, and placing the fly repelling article in an environment in which abatement of flies is desired, wherein the essential-oil composition provided herein is transferred from the fly repelling article into the environment, thereby repelling flies. In one embodiment, the fly repellent article is in the form of a cattle ear tag. In another embodiment, the fly repellent article is in the form or a strip or spiral that is hung in the locus to be treated, whereby wind or air movement causes transfer of the essential-oil composition from the fly repellent article into the atmosphere.

In another embodiment, provided herein is a method for repelling flies that includes dispersing a composition comprising at least 3% Lippia javanica oil in combination with one or more essential oils as provided herein into the atmosphere, whereby the flies are repelled from the locus in which the oils are dispersed. In one embodiment, the essential-oil composition is dispersed as a fine mist or spray. In one embodiment, the mist or spray is produced using a spray pump. In another embodiment, the mist is produced using an aerosol formulation of the composition provided herein. In another embodiment, the essential oil compositions provided herein are dispersed into the atmosphere by a diffuser. In one embodiment, the diffuser uses heat to accelerate the dispersion of the essential-oil composition into the atmosphere. In another embodiment, the diffuser using air and a jet nozzle or nebulizer to create a fine mist or spray of the essential-oil composition into the environment.

The fly repellent and treatment for killing flies may be prepared in various forms, such as an aqueous emulsion, an alcohol solution, a cream, a lotion, a powder, a gel, an oil, a rinse, a spray, a hair styling mousse, a shampoo, or a conditioner.

The fly repellent and treatment for killing flies may be delivered in various forms, such as a pump, a pump spray, a spray mist, an aerosol spray, a rinse, or liquid, for treating material, such as bedding material and/or fabric or for application to a subject.

5. Ants

Ants are members of the family Formicidae. There are almost 12,000 species of recorded species of ants, many of which occur in the sub-tropics and tropics (Wikipedia, the Free Encyclopedia, “Ant,” at URL http://en.wikipedia.org/wiki/Ant, January 2007). The more common pest species of ants include acrobat ants (Cremastogaster lineolata), argentine ants (Linepithema humile), army ants (Eciton burchelli), bullet ants (Paraponera spp.), carpenter ants (Camponotus spp.), cornfield ants (Lasius alienus), crazy ants (Paratrechina longicornis), false honey ants (Prenolepis imparis), fire ants (Solenopsis spp.), ghost ants (Tapinoma melanocephalum), jack jumper ants (Myrmecia pilosula), lawn ants (Iridomyrmex pruinosus var. analis), larger yellow ants (Acanthomyops interjectus), little black ants (Monomorium minimum), odorous house ants (Tapinoma sessile), pavement ants (Tetramorium caepitum), pharaoh ants (Monomorium pharaonis), red harvester ants (Pogonomyrmex barbatus), thief ants (Solenopsis molesta) and velvety tree ants (Liometopum occidentale).

Ants are one of the most common pests, both indoors and outdoors. Ants are generally considered as a nuisance pest, but some ants are capable of producing significant damage and can transmit disease. For example, carpenter ants can weaken wood in structures which could lead to their collapse. In addition, the colony building activities of ants can destroy landscaping. Ants are not normally considered a vector for disease. However, some ants have been implicated as carriers of antibiotic-resistant bacteria, including those of genera Acinetobacter, Enterobacter, Gemella, Klebsiella, Staphylococcus and Streptococcus (Moreira et al., Neotropical Entomology 34(6): 999-1006 (2005)). The stings of fire ants can threaten a person's health. Common insecticides for treating outdoor ant nests include permethrin as a liquid or as granules, carbaryl (e.g., Sevin®) as a liquid or granules, bifenthrin as granules, cyfluthrin as granules and acephate as a liquid. Common aerosol ready-to-use insecticides for treating ants found indoors include permethrin as an aerosol or a liquid, bifenthrin as liquid, cyfluthrin as a liquid, cypermethrin as an aerosol, deltamethrin as a liquid and tralomethrin as an aerosol or a liquid.

Provided herein are methods for killing ants. In one embodiment, provided herein is a method for killing ants, comprising the steps of topically applying to the ants an amount of a composition provided herein effective to kill ants, where the composition comprises: a) at least 3% Lippia javanica oil in combination with one or more essential oils; and b) a carrier.

In another embodiment, provided herein is a method for killing ants, comprising the steps of topically applying to the ants a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method to repel ants, comprising applying topically to locus prone to infestation with ants an amount of a composition provided herein effective to repel ants.

In another embodiment, the compositions provided herein are absorbed into or adsorbed onto a receptive or porous material, such as a pliable plastic, to produce an ant repellent article. In one embodiment, provided herein is a method for repelling ants that comprises absorbing into or adsorbed onto a receptive or porous material a composition comprising at least 3% Lippia javanica oil in combination with one or more essential oils as provided herein in an amount effective to repel ants to form an ant repelling article, and placing the ant repelling article in an environment in which abatement of ants is desired, such as in an area previously infested with ants, wherein the essential-oil composition provided herein is transferred from the ant repelling article into the environment, thereby repelling ants.

The ant repellent and treatment for killing ants may be prepared in various forms, such as an aqueous emulsion, an alcohol solution, a cream, a lotion, an oil, a powder, a gel, a rinse or a spray. The fly repellent and treatment for killing ants may be delivered in various forms, such as a pump, a pump spray, a spray mist, an aerosol spray, a rinse, or liquid, for treating a locus susceptible to infestation.

6. Mites

Some mites live their entire lives attached to the host, and there are two important groups. The Demodicidae are worm-like mites that live in the hair follicles of mammals, and the Psoroptidae and Sarcoptidae are mites that produce scab and mange. The more prevalent mites are the dust mites. World-wide, dust mites are the main causative agent of exacerbations of asthma. The World Health Organization has recognized domestic mite allergy as a universal health problem. The mites linked to asthma occur in domestic household dust. Adult house dust mites live in dust and their fecal matter, when inhaled, can elicit allergic reactions in certain individuals. Dust mites belong to the family Pyroglyphidae, and at least 13 species have been identified in house dust. The most common are Dermatophagoides pteronyssinus, Dermatophagoides farinae, Hirstia domicola, Malayoglyphus intermedius and Euroglyphus maynei. Mites are generally located in an environment that provides food and shelter with adequate humidity. Such areas in the home include, e.g., bed mattresses, carpets and upholstery. The dust mite population in the average bed may be greater than two million. The commercially available Acarex™ test kit may be used to assess house-dust mite levels in bedding, carpets or furniture fabric.

Mite control has generally been an extremely difficult task. The prior art solution is to remove as much of the habitat of mites as possible and to make what remains inhospitable to them. For example, physically encasing bedding and pillows is a quite effective mite control measure and is a commonly recommended approach. Regular weekly washing of fabrics in hot water at 130° F. (55° C.) or higher is effective for killing mites. Steam cleaning of carpets has also been used to reduce dust mite populations.

Prior art chemical substances used to kill mites include benzyl benzoate (e.g., Acarosan™), bromopol (e.g., Metsan™), pyrethroids and pirimiphos-methyl (e.g., Actellic™). These are used to some limited extent but their value is not proven at present. Long-term exposure effects to these prior art acaricides have not been documented, and the required repeated applications of these chemicals in the home, including bedroom areas, has been rejected by many people because the chemicals may prove to be toxic when used in confined spaces and on a regular basis.

Provided herein are methods for killing mites. In one embodiment, provided herein is a method for killing mites, comprising the steps of topically applying to the mites an amount of a composition provided herein effective to kill mites, where the composition comprises: a) at least 3% Lippia javanica oil in combination with one or more essential oils; and b) a carrier.

In another embodiment, provided herein is a method for killing mites, comprising the steps of topically applying to the mites a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for killing mites, comprising the steps of topically applying to a locus susceptible to infestation by mites, such as bedding material or carpeting, a killing amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method to repel mites, comprising applying topically to locus prone to infestation with mites, such as bedding material or carpeting, an amount of a composition comprising at least 3% Lippia javanica oil in combination with one or more essential oils provided herein effective to repel mites.

In another embodiment, the compositions provided herein are absorbed into or adsorbed onto a receptive or porous material, such as a pliable plastic, to produce a mite repellent article. In one embodiment, provided herein is a method for repelling mites that comprises absorbing into or adsorbed onto a receptive or porous material a composition comprising at least 3% Lippia javanica oil in combination with one or more essential oils as provided herein in an amount effective to repel mites to form a mite repelling article, and placing the mite repelling article in an environment in which abatement of mites is desired, such as in an area previously infested with mites, such as bedding material or carpeting, wherein the essential-oil composition provided herein is transferred from the mite repelling article into the environment, thereby repelling mites.

The mite repellent and treatment for killing mites may be prepared in various forms, such as an aqueous emulsion, an alcohol solution, a cream, an oil, a lotion, a powder, a gel, a rinse, a spray, a shampoo, a conditioner or a surfactant-blend suitable for softening fabrics. The mite repellent and treatment for killing mites may be delivered in various forms, such as a pump, a pump spray, a spray mist, an aerosol spray, a rinse, or liquid, for treating a locus susceptible to infestation.

In another embodiment, provided herein is a method for killing and/or repelling mites that includes dispersing an essential-oil composition provided herein into the atmosphere, whereby the mites are repelled from the locus in which the oils are dispersed or whereby the essential-oil composition is dispersed and comes into contact with the mites, thereby killing the mites. In one embodiment, the essential-oil composition is dispersed as a fine mist or spray. In one embodiment, the mist or spray is produced using a spray pump. In another embodiment, the mist is produced using an aerosol formulation of an essential-oil composition provided herein. In another embodiment, an essential-oil composition provided herein is dispersed into the atmosphere by a diffuser. In one embodiment, the diffuser uses heat to accelerate the dispersion of the essential-oil composition into the atmosphere. In another embodiment, the diffuser using air and a jet nozzle or nebulizer to create a fine mist or spray of the essential-oil composition into the environment.

In another embodiment, a method of killing mites is provided, the method comprising washing the clothing or bedding materials infested with mites in hot water, and adding an essential-oil composition provided herein to the wash cycle or rinse cycle or both, thereby killing the mites. In one embodiment, at least 1 ounce of an essential-oil composition provided herein is added to the wash or rinse cycle.

7. Ticks

Ticks are of the parasitic Acarines, a group that includes ticks, chiggers and mites. Ticks are arachnids like mites, spiders and scorpions. Ticks are blood-feeding external parasites of mammals and birds. There are more than 800 species worldwide. However, there are two well-established families of ticks—the Ixodidae, known as the hard ticks, and the Argasidae, known as the soft ticks. Ticks generally remain attached to the host for a relatively short period of time. Ticks are vectors of disease to both humans and animals, transmitting viruses, bacteria, spirochetes, protozoa and rickettsiae (zoonotic pathogens including the genera Rickettsiae, Ehrlichia, Orientia, and Coxiella). Some of the diseases transmitted by ticks include Lyme disease, epidemic typhus, Rocky Mountain spotted fever, Colorado tick fever, relapsing fever, tularemia, babesiosis and ehrlichiosis. Lyme disease is by far the most often reported tick-borne disease in humans in the United States.

Provided herein are methods for killing ticks on a subject. In one embodiment, provided herein is a method for killing ticks on a subject, comprising the steps of topically applying to the subject an amount of the composition provided herein effective to kill ticks, where the composition comprises: a) at least 3% Lippia javanica oil in combination with one or more essential oils; and b) a carrier.

In another embodiment, provided herein is a method for killing ticks on a subject, comprising the steps of topically applying to the subject a therapeutically effective amount of the composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided herein is a method for killing ticks on a subject, comprising the steps of topically applying to the subject a therapeutically effective amount of a composition provided herein, wherein the composition comprises: a) at least 5% Lippia javanica oil in combination with one or more essential oils; b) a non-essential oil; and c) an alcohol, wherein the composition is applied in the form of an oil, a lotion, a cream or a foam.

In one embodiment, a composition provided herein is applied to the subject for a time sufficient to kill the ticks. In one embodiment, the composition is applied for at least 30 minutes. In one embodiment, the composition is applied for about 20 minutes. In one embodiment, the composition is applied for about 10 minutes. In one embodiment, the composition is applied for about 5 minutes.

In another embodiment, provided herein is a method for killing ticks in a locus where such tick control is desired. In one embodiment, provided herein is a method for killing ticks, comprising the steps of topically applying to the ticks or the environment or locus exhibiting tick infestation an amount of a composition provided herein effective to kill ticks, where the composition comprises: a) at least 3% Lippia javanica oil in combination with one or more essential oils; and b) a carrier.

In another embodiment, provided herein is a method for killing ticks, comprising the steps of topically applying to the tick or the locus infested therewith a therapeutically effective amount of a composition provided herein, wherein the composition comprises: a) at least 5% Lippia javanica oil in combination with one or more essential oils; b) a non-essential oil; and c) an alcohol. Optionally, the composition may contain isopropyl palmitate. Optionally, the composition also may contain a preservative and/or an antioxidant.

In another embodiment, provided is a method for killing ticks in a carpet, comprising dispersing or mixing a composition provided herein with one or more dispersing powders selected from among an inorganic sulfate, bicarbonate or chloride, an aluminum silicate clay to form a powdered dispersion of the composition, applying the powdered dispersion of the composition uniformly to the carpet and thereafter removing it from the carpet. Methods of making a carpet powder are known in the art (for example, see U.S. Pat. No. 4,873,000). In addition to powders, in one embodiment provided herein is a foam composition for application to a carpet for killing ticks. The composition includes an essential-oil blend disclosed herein formulated in a friable foam carpet formulation, such as the formulation described in U.S. Pat. No. 6,315,949.

In another embodiment, provided is a method of killing ticks in a vacuum cleaner, comprising dispersing or mixing a composition provided herein with rock salt or a granule, such as granular attapulgite or heat expanded, granular, screened vermiculite to form a granular dispersion of the composition, and placing the granular dispersion of the composition into the dust collecting bag of a vacuum.

In another embodiment, provided herein is a method to repel ticks, comprising applying topically to a subject susceptible to tick infestation an amount of a composition provided herein effective to repel but not kill ticks.

In another embodiment, the method comprises adding a combination of essential oils as provided herein in an amount effective to repel ticks to a shampoo, a conditioner, a hair spray, a hair rinse, a styling gel or other haircare product, and applying the haircare product to the hair of a subject, whereby ticks are repelled. The repellent compositions also are effective as a repellent when applied to clothing, furniture, upholstery, carpeting and bedding.

The tick repellent compositions provided herein also are absorbed into or adsorbed onto a receptive or porous material, such as a pliable plastic, that may be formed into a loop, e.g., as in a tick collar. In one embodiment, provided herein is a method for repelling ticks that comprises absorbing into or adsorbed onto a receptive or porous material a combination of essential oils as provided herein in an amount effective to repel ticks to form a tick repelling article, and placing the tick repelling article on the subject to repel ticks.

The tick repellent and treatment for killing ticks may be prepared in various forms, such as an aqueous emulsion, an alcohol solution, a cream, a powder, an oil, a gel, a rinse, a spray, hair styling mousse, shampoo, or conditioner.

The tick repellent and treatment for killing ticks may be delivered in various forms, such as a pump, a pump spray, a spray mist, an aerosol spray, a rinse, a liquid or a dip for brushes, combs, and for treating material, such as bedding material and/or fabric or for application to a subject. The tick repellent and treatment for killing ticks is applied once and may be repeated at intervals including weekly, every two weeks, once a month or every other month.

In another embodiment, a method of killing ticks is provided, the method comprising washing the clothing, upholstery or bedding materials infested with ticks in hot water, and adding an essential-oil composition provided herein to the wash cycle or rinse cycle or both, thereby killing the ticks. In one embodiment, at least 1 ounce of an essential-oil composition provided herein is added to the wash or rinse cycle.

6. Articles of Manufacture

In some embodiments, an essential-oil compositions provided herein and formulations thereof are packaged as articles of manufacture containing a packaging material, within the packaging material an essential-oil composition provided herein and formulations thereof, which is effective for repelling or killing ectoparasites or pests, and a label that indicates that the essential-oil composition provided herein or formulation thereof, is used for repelling or killing ectoparasites or pests.

The articles of manufacture provided herein contain packaging materials. Packaging materials for use in packaging products are well known to those of skill in the art (see, e.g., U.S. Pat. Nos. 5,323,907, 5,052,558 and 5,033,252). Examples of packaging materials include, but are not limited to, blister packs, bottles, tubes, vials, jars, containers, foil packets, aerosol bottles and devices, and any packaging material suitable for a selected formulation and intended mode of administration and treatment. A wide array of formulations of the compositions provided herein and formulations thereof are contemplated.

In certain embodiments, the compositions are presented in a pack or dispenser device which may contain one or more unit dosage forms containing a composition provided herein. The pack may, for example, contain metal or plastic foil, such as a blister pack. The pack or dispenser device may be accompanied by instructions for administration. Compositions provided herein also may be prepared, placed in an appropriate container, and labeled for treatment of an indicated condition, such as repelling or killing ectoparasites or pests. In some embodiments, the composition is provided in a portable personal aerosol container or spray device, such as described in U.S. Pat. No. 6,415,992.

In another embodiment, provided herein are containers in which the compositions of the subject invention are sold and/or distributed. In one embodiment, these containers include the essential-oil formulations provided herein and have instructions for the use of the essential-oil compositions provided herein for repelling or killing ectoparasites and/or pests. In another embodiment, the containers are glass, metal or plastic (or other appropriate inert material). In one embodiment, the formulation is concentrated. In another embodiment, the formulation is prepared for immediate use. In one embodiment, the instructions pertain to the use of the blend of essential oils provided herein for repelling or killing ectoparasites and/or pests. In one embodiment, the instructions for use are written on the outside of the container.

7. Kits

In another embodiment, the present invention also provides kits for treating ectoparasite and/or pest infestations. In other embodiments, the present invention provides kits for repelling ectoparasites and/or pests. In other embodiments, the present invention provides kits for detecting and repelling or killing ectoparasites and/or pests. In various embodiments the kits include a composition of the present invention in a package or other enclosure. In other embodiments the kits further include a flea comb or a “nit comb” to assist in removing ectoparasites and/or pests and their eggs from hair. The “nit comb” is an ordinary comb for ordering hair by passing it through the hair. For example the LiceMeister® (National Pediculosis Association, Inc., Newton, Mass.), ACU-MED® Lice Comb (Health Enterprises, N. Attleboro, Mass.), and MEDI-SWEEP® Lice Comb (Classic Products, Oxnard, Calif.) are exemplary lice combs to be included in the kit. In one embodiment, the package is a box that contains the composition provided herein, instructions for use, and optionally an applicator or a comb. In another embodiment, the kit is provided in a wrapping (such as a plastic) that surrounds the kit. In one embodiment, the comb is provided inside the package, but also may be attached to the outside of the package. In other embodiments the kits include shower caps. In other embodiments the kit also contains instructions that describe how to use the items included in the kit to kill or repel ectoparasites and/or pests.

In another embodiment, the present invention also provides kits comprising an essential-oil composition provided herein. In one embodiment, the kit includes one or more essential-oil compositions provided herein and a pump sprayer for application of the composition(s). In another embodiment, the kit includes one or more essential-oil compositions provided herein and an applicator for applying the composition(s) to the hair or skin. In another embodiment, the kit includes one or more essential-oil compositions provided herein and an applicator for dispensing or applying the composition(s) to ectoparasites and/or pests.

In another embodiment, the kit includes one or more essential-oil compositions provided herein and a diffuser to dispense the composition(s) into the atmosphere. In one embodiment, the kit includes one or more essential-oil compositions provided herein and a diffuser, such as any of the diffusers described in U.S. Pat. Nos. 6,645,436, 6,090,349 and 4,762,275. In another embodiment, the kit includes one or more essential-oil compositions provided herein and an absorbent carrier for accepting and diffusing the essential oil compositions. In another embodiment, the kit includes one or more essential-oil compositions provided herein and a porous diffusing material.

In another embodiment, provided is a kit comprising an essential-oil composition provided herein, a testing assay for the presence of ectoparasites and/or pests, and instructions for using the provided essential-oil composition to kill ectoparasites and/or pests. The assays generally detect the presence of ectoparasite or pest feces. In one embodiment, the kit comprises an assay to the presence of mites. In one embodiment, the testing assay for mites is an immunoassay, such as described by Mistrello et al. (J Med Entomol. 35(2):143-147 (1998)). In another embodiment, the testing assay for mites is a guanine test or the Acarex™ test kit, sold and distributed by Fisons Pharmaceuticals (Rochester, N.Y.) in the United States (e.g., see Haouichat et al., Indoor and Built Environment 10:109-115 (2001)). The mite test assays generally are “dipstick-type” tests similar to those sold in drugstores for glucose.

### F. Evaluation of the Activity of the Compositions

Standard physiological, pharmacological and biochemical procedures and assays are available for testing the compositions provided herein (e.g., see WHO (World Health Organization) 1981, “Instructions for determining the susceptibility or resistance of body lice and head lice to insecticides,” WHO/VBC/81.808; US Army Environmental Hygiene Agency, “Procedures for the Diagnostic Dose Resistance Test Kits for Mosquitoes, Body Lice and Beatle Pests of Stored Products,” TG No. 189 (1992) and Picollo et al., J Medical Entomology 37(5): 721-725 (2000), the disclosure of each of which is incorporated in its entirety by reference herein). In vitro and in vivo assays known in the art may be used to evaluate the activity of the compositions provided herein. Several formulations have been produced and tested in such assays, as described below, and shown to be effective in knocking down and/or killing and/or repelling ectoparasites and/or pests.

The compositions and methods provided herein will be further illustrated in the following, non-limiting examples. The Examples are illustrative of various embodiments only and do not limit the claimed invention regarding the materials, conditions, weight ratios or process parameters recited herein.

### EXAMPLES

### Example 1

Preparation of Lippia javanica Compositions

Several formulations comprising Lippia javanica essential oil in combination with one or more other plant essential oils were prepared. The composition of these exemplary formulations is shown in Table 1.

Each essential-oil composition was prepared by combining the essential oils with thorough mixing, using an overhead paddle mixer at a speed of 250 rpm. This resulted in a clear solution. The color and aroma of each formulation were recorded, the results of which are shown in Table 2.

TABLE 1
Lippia javanica Compositions

| | Formula #: | | | | | | | | |
|---|---|---|---|---|---|---|---|---|---|
| | A | B | C | D | E | F | G | H | I |
| Ingredient | Wt % of Essential Oil | | | | | | | | |
| Lippia javanica oil | 14.13 | 21.74 | 43.48 | 11.11 | 66.67 | 50 | 30 | 30 | 14.26 |
| Lemongrass oil | 22.97 | 21.74 | | | | 25 | | | 23.28 |
| Rosmarinum officinalis oil | 9.19 | 7.25 | | | | | 20 | | 9.03 |
| (rosemary oil) | | | | | | | | | |
| Lavendula augustifolia oil | 16.61 | 14.49 | | | | | 20 | 20 | 17.33 |
| (lavender oil) | | | | | | | | | |
| Melaleuca alternifolia oil | 35.33 | 33.33 | | | | | | 20 | 36.1 |
| (tea tree oil) | | | | | | | | | |
| Thymus vulgaris oil | 1.76 | 1.45 | | | | | | | |
| (thyme leaf oil) | | | | | | | | | |
| Eucalyptus oil | | | | | 33.33 | | | 30 | |
| Lemon oil | | | | | | 25 | | | |
| Petitgrain oil | | | | 44.44 | | | 30 | | |
| Cinnamon oil | | | 21.74 | | | | | | |
| Lovage oil | | | | 11.11 | | | | | |
| Tagete minuta oil | | | 34.78 | 11.11 | | | | | |
| (marigold oil) | | | | | | | | | |

TABLE 2
Characteristics of Exemplary Essential Oil Compositions

| Formulation | Color | Aroma |
|---|---|---|
| A | amber to light | strong citrus smell |
| | orange | |
| B | amber to light | strong citrus smell |
| | orange | |
| C | light orange | peppery, faint floral notes |
| D | dark yellow | sharp, citrus, faint floral notes |
| E | orange-yellow | strong pine, peppery notes |
| F | light orange | light citrus, sweet smell |
| G | deep orange | soft, sweet delicate, citrus and floral notes |
| H | dark yellow | strong pine (not over-powering), some soft |
| | | notes |
| I | light orange | strong citrus smell, faint floral notes |

### Example 2

Preparation of Formulations of the Essential Oil Compositions

Formulations comprising the Lippia javanica essential-oil composition provided herein were prepared. In addition, control formulations of Lippia javanica essential oil alone and formulations with no Lippia javanica essential oil also were prepared. The formulations were prepared by combining the appropriate essential oils with thorough mixing, using an overhead paddle mixer at a speed of 250 rpm. This resulted in a clear amber to deep-orange solution. In a separate container, the coconut oil was liquefied by heating to 25° C. while stirring with an overhead stirrer revolving at a speed of 250 rpm, resulting in a clear colorless liquid. If present, the soybean oil was added to the coconut oil at this point. The blend of essential oils was added to the coconut oil or coconut oil/soybean oil blend with constant mixing at a reduced speed (50-200 rpm). The resulting oil mixture was a clear amber to orange colored solution. The tocopherol then was added to the oil mixture with constant mixing, resulting in a clear amber to orange solution. In a separate container, the isopropyl palmitate was added to the denatured alcohol, resulting in a clear colorless solution. The cyclo-methicone was added to this alcohol solution, resulting in a milky solution. The propylparaben then was dissolved in the alcohol solution, resulting in a milky solution. The alcohol solution then was added to the oil mixture, resulting in a yellow to orange solution. Exemplary formulations are shown in Table 3.

TABLE 3
Formulations

| | Formula #: | | | | |
|---|---|---|---|---|---|
| | 1 | 2 | 3 | 4 | 5 |
| Ingredient | % Wt of Formulation | | | | |
| Lippia javanica oil | 50 | — | 8 | 15 | — |
| Lemongrass oil | — | 17 | 13 | 15 | 15 |
| Rosmarinum officinalis oil (rosemary oil) | — | 5.2 | 5.2 | 5 | — |
| Lavendula augustifolia oil (lavender oil) | — | 10.4 | 9.4 | 10 | 10 |
| Melaleuca alternifolia oil (tea tree oil) | — | 23 | 20 | 23 | 23 |
| Thymus vulgaris oil (thyme leaf oil) | — | 1 | 1 | 1 | 1 |
| Tagete minuta oil | — | — | — | — | 10 |
| (marigold oil) | | | | | |
| Levisticum officinalis (lovage) oil | — | — | — | — | 9 |
| Isopropyl palmitate | 5 | 6 | 10 | 5 | 3 |
| Cocos nucifera oil (coconut oil) | 5 | 10 | 10 | 5 | 3 |
| Soybean oil | 23 | — | — | 4 | 3 |
| Cyclomethicone | 4 | 5 | 6.4 | 4 | 3 |
| SD Alcohol 40-B (denatured alcohol) | 10 | 10 | 10 | 10 | 10 |
| Propylparaben | 1 | 2 | 2 | 1 | 2 |
| Tocopherol | 2 | 10.4 | 5 | 2 | 8 |

When applied to the forearm, the formulations were perceived as being “oily” but after rinsing with water did not leave a greasy or oily film.

### Example 3

### Demonstration of the Effectiveness of Compositions in Treating Human Head Lice

Formulations prepared according to Example 2 were tested for their effectiveness in treating head lice.

### Specimen Collection

The head lice were harvested from infested individuals that were not treated with a pediculicidal shampoo within the previous three weeks. Participating study staff washed their hands with antibacterial soap and rinsed in clean tap water, followed by a rinse in 70% alcohol to remove any possible contaminants. Using a lighted 10× magnifying lamp, the technicians collected head lice from infested individuals and placed them into a Petri dishes (60×15 mm) each containing a 100% cotton cloth disk that was dampened with filtered water to prevent the lice from dehydrating. Specimens were examined through a microscope for viability, intact legs and antennae. The viable intact specimens were kept in an incubator at a temperature of 80° F. (±5°) and relative humidity of 80% (±10%). Approximately one-half hour prior to testing, the Petri dishes containing the viable lice specimens were removed from the incubator to acclimate to testing conditions (a temperature of 76° F. (±10°) and relative humidity of 68% (±15%). Test specimens that were in incubation in the Petri dishes for more than 5 hours were not used for testing. In most cases, lice harvested from invested individuals were used directly in the test assay at testing conditions (a temperature of 76° F. (±10°) and relative humidity of 68% (±15%). The head lice used in the assay were collected and pooled from 7 participants. The lice were of different gender and stage of development; the male and female adults and nymphs were distributed evenly between test samples and the controls.

### Pediculicidal Assay

One large cotton cheese cloth towel was washed with a small quantity of Prell® shampoo (Prestige Brands, Inc., Irvington, N.Y.), a non-medicated anionic shampoo. The towels were thoroughly rinsed and machine dried. The cotton towel was cut into disks to fit into the bottom of the sterile Petri dish. One mL of the test composition was distributed evenly on each of the cloth disks and allowed to thoroughly impregnate the cloth. The amount of composition used in the assay produced a wet surface upon which the lice remain in intimate contact during the assay.

Lice specimens were carefully and gently transferred to the test dishes using entomological forceps to avoid physical damage to the lice. The lice were observed with a microscope for eight (8) hours at intervals of 5 minutes. The following parameters were observed and recorded.

1) Behavior: Movement of the lice, which included agitation, running for cover, trying to hide, trying to escape, walking about, walking with unsteady gait, stumbling, disorientation, and tremors.

2) Incapacitation: Inability to walk, only occasional tremors or twitching of legs or antennae. This is a stage from which lice rarely or never recover. At this stage they are described as moribund.

3) Death: When all movement of limbs, antennae and peristalsis of gut ceases. The definition of death in lice exposed to toxicants is difficult to determine. In many instances, individual lice may show no sign of life except an occasional twitch of an antenna or claw. At this stage, they are harmless, cannot feed, infest another person, or lay eggs. Time of death was recorded when all movements had ceased.

For Formulation 1, which included 50% Lippia javanica essential oil, the lice were walking about and trying to hide after 10 minutes of exposure. After 20 minutes of exposure, some were standing still, some were struggling, and 10% were dead. By 30 minutes, all surviving lice were struggling and 20% were dead. After 60 minutes of exposure, all surviving lice were struggling but 80% were still moving around (only 20% mortality).

For Formulation 2, which included a combination of lemongrass oil, Rosmarinum officinalis oil, Lavendula augustifolia oil, Melaleuca alternifolia oil and Thymus vulgaris oil, the lice were walking about and trying to hide after 10 minutes of exposure, but all lice were alive. After 20 minutes of exposure, some surviving lice were standing still, some were struggling, and between 0-20% of the lice were dead. By 30 minutes exposure time, all of the surviving lice were struggling and 20% were dead. After 40 minutes of exposure, all surviving lice were struggling and 80% to 100% were dead. After 60 minutes of exposure, all surviving lice were struggling and 80% to 100% of the lice were dead. By 90 minutes exposure time, 100% of the lice were dead.

Formulations 3 and 4 included Lippia javanica essential oil in combination with lemongrass oil, Rosmarinum officinalis oil, Lavendula augustifolia oil, Melaleuca alternifolia oil and Thymus vulgaris oil. All lice were trying to hide upon application of the formulation and were disabled almost as soon as they were exposed to the formulation. At 5 minutes post exposure, there was no movement in any of the lice, and 40% were dead. After 10 minutes of exposure, 100% of the lice were dead.

Formulation 5 did not include Lippia javanica essential oil. It included a blend of Levisticum officinalis (lovage) oil, Tagete minuta (marigold) oil, lemongrass oil, Lavendula augustifolia oil, Melaleuca alternifolia oil and Thymus vulgaris oil. After 10 minutes of exposure, the lice were running around agitated, but all lice were alive. After 20 minutes of exposure, the lice were standing still, exhibiting some movement and trembling, but all lice were alive. By 30 minutes exposure time, all of the surviving lice were still and not moving and 0% to 20% were dead. After 40 minutes of exposure, all surviving lice were struggling and 20% to 60% were dead. After 60 minutes of exposure, all surviving lice were struggling and 60% to 100% of the lice were dead. By 90 minutes exposure time, 100% of the lice were dead. The control lice had 0% mortality of lice in 90 minutes.

Thus, Formulation 1, which included 50% Lippia javanica essential oil, was only 20% effective in killing head lice when applied for 30 minutes. Formulation 2, which included a combination of lemongrass oil, Rosmarinum officinalis oil, Lavendula augustifolia oil, Melaleuca alternifolia oil and Thymus vulgaris oil was only 20% effective in killing head lice when applied for 30 minutes. Formulation 5, which included a blend of Levisticum officinalis (lovage) oil, Tagete minuta (marigold) oil, lemongrass oil, Lavendula augustifolia oil, Melaleuca alternifolia oil and Thymus vulgaris oil, was 20% effective in killing head lice when applied for 30 minutes. Exposure of lice to Formulations 3 and 4, which included Lippia javanica essential oil in combination with of lemongrass oil, Rosmarinum officinalis oil, Lavendula augustifolia oil, Melaleuca alternifolia oil and Thymus vulgaris oil, resulted in almost complete immobilization of the lice, followed by substantial mortality. Mortality was 100% following 5 to 10 minute exposure.

Thus, formulations that included Lippia javanica oil alone did not kill 100% of the lice at exposure times of 30 minutes. Further, combinations of essential oils that did not include Lippia javanica did kill head lice, but did not demonstrate 100% mortality within 30 minutes. The addition of Lippia javanica essential oil to such compositions demonstrated a synergistic effect of Lippia javanica essential oil with other essential oils in killing head lice, resulting in 100% knockdown within 5 minutes of application, and 100% mortality in 10 minutes or less of application. Thus, formulations including Lippia javanica essential oil have an increased killing efficiency compared to comparable formulations of essential oils that do not include Lippia javanica essential oil.

The formulations comprising Lippia javanica essential oil in combination with other essential oils resulted in almost complete immobilization of the lice, followed by substantial mortality. Mortality was 100% following 5-10 minute exposure. Thus, the formulations comprising Lippia javanica essential oil in combination with other essential oils showed excellent pediculicidal activity against head lice. The formulations comprising Lippia javanica essential oil in combination with other essential oils exhibited 100% kill rate following 5-10 minutes exposure demonstrating high efficacy after a very short application time.

### Example 4

### Demonstration of the Effectiveness of Compositions in Treating Cat Fleas

An exemplary formulation comprising Lippia javanica essential oil in combination with other essential oils was tested to determine the efficacy of a direct spray formulation against adult cat fleas on carpet. The formulation tested was Formulation 3.
METHOD: Each test sample was applied directly to fleas, and five replicate tests were performed. Knockdown counts were recorded after 1 hour exposure. Mortality counts were taken at approximately 24 hours after exposure.
SPRAYERS: The test sample was dispensed using a hand pump sprayer. The application amount was two pump activations per test replicate.
CONTAINERS: Holding containers were five-gallon plastic buckets approximately 25.4 cm inside diameter bottom, approximately 29.3 cm inside diameter top, and approximately 36.8 cm high.
TEST SUBSTRATE: Carpet discs, approximately 25.4 cm in diameter were cut from approximately 1 cm pile nylon carpet which fit within the holding containers.
FORMULATIONS: The test samples were maintained at ambient temperature and humidity until commencing the study.
APPLICATION RATE: The application rate was two activations of the pump mechanism per replicate. The rate of delivery is 1.5 g per activation. The actual amount applied per test replicate was recorded. To determine the actual application rate, a Mettler balance was used to weigh the test formulation before and after each test replicate. The weight of the spray delivered per test replicate was calculated from the weights of each sample before and after treatment.
PREPARATION AND HANDLING OF FLEAS: Twenty cat fleas were aspirated from the rearing container and placed on each of the carpet circles. Five treatment replicates were sprayed with the test formulation, and five replicates served as the untreated controls. Each of the test carpet circles were then sprayed with the test compound. Control carpet circles received no treatment.
TREATMENT OF TEST FLEAS: Five replicates of fleas were placed in a Peet Grady chamber (one at a time) and sprayed with two activations with the test product held approximately at the rim of the plastic pail. The test sample was weighed before and after each replicate to determine the weight of compound applied.

All carpets were held in the laboratory under ambient temperature and humidity conditions with an approximately 9:15 light:dark cycle for 24 hours. Final mortality counts were made approximately 24 hours after application.
TREATMENT OF CONTROL FLEAS: Each replicate of control fleas was subjected to the same procedures outlined above with the exception that they were not treated. The untreated controls were housed in the same area as those treated fleas for the duration of the prescribed observation periods.
MORBIDITY AND MORTALITY: Fleas were observed at approximately 1 hour after exposure to observe knockdown and approximately 24 hours after exposure to observe mortality. The +1 hour observation involved gently blowing on the carpet and observing flea activity. Counts were approximate only and give an indication of effect of the compound. At 24 hours after exposure, the carpet circles were thoroughly and destructively searched to determine the final mortality. The fleas were categorized as alive, moribund (unable to hop), or dead. The total number of moribund fleas was combined with the number of dead fleas to calculate percent mortality. Any fleas not found were excluded from the data analysis.
DATA ANALYSIS: Data analysis was performed as appropriate. Average 24-hour mortality was calculated, after adjusting for control mortality using Abbott's formula:

PT=(PO−PC/100−PC)×100

where PT is adjusted mortality, PO is observed mortality and PC is control mortality (e.g., see Abbot, “A method for computing the effectiveness of an insecticide,” J Econ Entomol 18:265-267 (1925)). Abbott's formula adjusts for mortality not associated with insecticide treatment such as the natural mortality in an untreated control group or mortality occurring from a blank spray used as a control. Adjusted % mortality=% alive in control−% alive in treatment×100% alive in control.
RESULTS: The mortality counts were converted to percentages and then averaged to obtain mean values. Exposure to Formula 3 for 1 hour resulted in 77.7% knockdown and 100% mortality 24 hours after exposure. Thus, Formulation 2 was judged effective against cat fleas.

### Example 5

### Demonstration of the Effectiveness of Compositions in Killing Houseflies

An exemplary formulation comprising Lippia javanica essential oil in combination with other essential oils was tested to determine the efficacy of a direct spray formulation against houseflies (Musca domestica). The formulation tested was Formulation 3.
TEST ORGANISM: House flies, Musca domestica, were tested. Five replicates of 10 adult house flies were used.
TREATMENT CHAMBER AND CONTAINER: Treatment was done in a 6×6×6 foot Peet Grady Chamber. Test containers were cardboard cylinders, approximately 3 inches in diameter by 2 inches long, screened at both ends with a side entry hole covered with a rubber dam.
METHODS: The test formulation was applied directly to 5 cages of 10 house flies each. Knockdown counts were taken at 5 minutes, 15 minutes and 1 hour post application. Mortality readings were taken 24 hours post-treatment.
PREPARATION AND HANDLING OF THE HOUSEFLIES: Ten house flies were aspirated into each of five cages. Five additional cages were set up for untreated controls.
TREATMENT OF HOUSEFLIES: Each of the cages were placed in the Peet Grady chamber and directly sprayed with the test compound. The five control cages were handled in the same manner as the treated cages except they were not treated. The control cages were placed in the treatment chamber for 10 minutes before treatment to confirm that the area was not contaminated.

The house flies were maintained in the laboratory under ambient temperature and humidity conditions with a 15:9 light:dark cycle for 24 hours. The flies were provided with 10% sucrose administered in a soaked cotton ball.
OBSERVATION OF KNOCKDOWN AND MORTALITY: House flies were observed for knockdown at 5 minutes, 15 minutes and 1 hour post-application. Knockdown was recorded for any house flies that were unable to fly. Mortality counts were taken 24 hours post-application.
DATA ANALYSIS: Data analysis was performed as appropriate. Abbot's formula, as described above, was used to correct for any mortality that occurred in the control samples. The knockdown counts and the 24-hour mortality counts were converted to percentages, and then averaged to obtain mean values.
RESULTS: There was 4.0% mortality in the controls. However, correction for this mortality was not necessary as treatment with the test sample resulted 100% mortality. No statistical analysis was required as treatment with the test sample produced 100% mortality at 24 hours.

Treatment with Formulation 3 resulted in 76% knockdown at +5 minutes application, and provided 100% knockdown at +15 minutes and at +60 minutes post-application. Treatment with Formulation 3 resulted in 100% mortality at +24 hour post-treatment.

Thus, the Formulation 3 was considered completely effective against houseflies under these test conditions.

### Example 6

### Demonstration of the Effectiveness of Compositions in Killing Mosquitoes

An exemplary formulation comprising Lippia javanica essential oil in combination with other essential oils was tested to determine the efficacy of a direct spray formulation against mosquitoes. The formulation tested was Formulation 3.
TEST ORGANISM: Aedes aegypti (yellowfever mosquitoes) were tested. Five replicates of 10 adult female yellow-fever mosquitoes (3-8 days old) were used.
TREATMENT CHAMBER AND CONTAINER: Treatment was performed in a 6×6×6 foot Peet Grady Chamber. Test containers were cardboard cylinders, approximately 3 inches in diameter by 2 inches long, screened at both ends with a side entry hole covered with a rubber dam.
TEST SAMPLE HANDLING: The test samples was stored at ambient temperature and humidity until performing the test.
PREPARATION AND HANDLING OF THE MOSQUITOES: Ten mosquitoes were aspirated into each of five cages. Five cages were set up for untreated controls.
TREATMENT OF MOSQUITOES: Each of the cages was placed in the Peet Grady chamber and directly sprayed with the test compound.

The five control cages were handled in the same manner as the treated cages except they were not treated. The control cages were placed in the treatment chamber for 10 minutes before treatment to confirm that the area was not contaminated.

The mosquitoes were maintained in the laboratory under ambient temperature and humidity conditions with a 15:9 light:dark cycle for 24 hours. The mosquitoes were provided with 10% sucrose administered in a soaked cotton ball.
OBSERVATION OF KNOCKDOWN AND MORTALITY: Mosquitoes were observed for knockdown at 5 minutes, 15 minutes and 1 hour post-application. Knockdown was recorded for any mosquitoes that were unable to fly. Mortality counts were taken 24 hours post-application.
DATA ANALYSIS: Data analysis was performed as appropriate. Abbot's formula, as described above, was used to correct for any mortality that occurred in the control samples. The knockdown counts and the 24-hour mortality counts were converted to percentages, and then averaged to obtain mean values.
RESULTS: There was no mortality observed in the control samples. Therefore correction of test mortality using Abbott's formula was not necessary. No statistical analysis was required because the test sample produced 100% knockdown at +5, +15 and +60 minutes post-application and 100% mortality at 24 hours post-application.

Application of Formulation 3 resulted in 100% knockdown within the first 5 minutes after application and 100% mortality 24 hours post-application. Thus, this formulation was considered completely effective against this species of mosquito under these test conditions.

### Example 7

### Demonstration of the Effectiveness of Compositions in Killing Ants

An exemplary formulation comprising Lippia javanica essential oil in combination with other essential oils was tested to determine the efficacy of a direct spray formulation against ants. The formulation tested was Formulation 3.

A trail of argentine ants was sprayed with Formulation 3 using a pump sprayer. The ants appeared to be immobilized on contact with Formulation 3. No movement was observed in the treated ants after 1 minute of application of the formulation. Application of Formulation 3 resulted in 100% knockdown within the first minute after application and 100% mortality within 5 minutes post-application. Thus, this formulation was considered completely effective against this species of ants under these test conditions.

### Example 8

### Solid Granule Formulation

A course solid granule formulation of an essential-oil composition provided herein was prepared. In a small bowl or container, 2 grams of Formulation 3 was added to 98 grams of course salt (such as table salt, sea salt or Epsom Salt). The essential-oil composition and salt was mixed well to evenly distribute the essential-oil composition on the salt granules. The resulting granular formulation was packaged in a glass container and sealed. The solid granule formulation was placed in the dust bag of a vacuum to kill ectoparasites collected by the vacuum.

### Example 9

### Dry Powder Formulation

A dry powder formulation of an essential-oil compositions provided herein was prepared. In a small bowl or container, 1 gram of Formulation 3 was added to 99 grams of sodium bicarbonate. The essential-oil composition was added slowly in small aliquots to the sodium bicarbonate and mixed well to evenly distribute the essential-oil composition on the powdered sodium bicarbonate. The resulting dry powder formulation was packaged in a glass container and sealed. The dry powder was sprinkled on carpeting to kill fleas, lice, mites or other ectoparasites or pests that may be present, and after waiting for 30 minutes, the powder was removed from the carpet using a vacuum.

### Example 10

### Sprayable Formulation

Sprayable formulation of the compositions provided herein were made. For one formulation, the formulation was prepared by combining 13% by weight lemongrass oil, 8% Lippia javanica oil, 9.4% Lavendula augustifolia (lavender) oil, 20% Melaleuca alternifolia (tea tree) oil, 5.2% Rosmarinum officinalis (rosemary) oil and 1% Thymus vulgaris (thyme) oil with thorough mixing, using an overhead paddle mixer at a speed of 250 rpm. This resulted in a clear light orange solution. In a separate container, 10% coconut oil was liquefied by heating to 25° C. while stirring with an overhead stirrer revolving at a speed of 250 rpm, resulting in a clear colorless liquid. The blend of essential oils was added to the coconut oil with constant mixing at a reduced speed (50-200 rpm). Five percent tocopherol then was added to the oil mixture with constant mixing. In a separate container, 10% isopropyl palmitate was added to 10% SD 40-B denatured alcohol, resulting in a clear colorless solution. To this alcohol solution was added 6.4% cyclomethicone and 2% propylparaben with constant stirring. The alcohol solution then was added to the oil mixture, resulting in a sprayable formulation. The composition was placed in a pump spray bottle, which produced a fine mist of the formulation upon actuation of the pump. This formulation is appropriate for topical application to a subject, to an ectoparasite or pest and for a locus to be treated, such as fabric, clothing, upholstery, carpeting and bedding.

The foregoing description is intended to illustrate various aspects of the present invention. It is not intended that the examples presented herein limit the scope of the present invention. The invention now being fully described, it will be apparent to one of ordinary skill in the art that many changes and modifications may be made thereto without departing from the spirit or scope of the appended claims.

I claim:

1. A composition for killing or repelling ectoparasites and/or pests, comprising at least 3% Lippia javanica essential oil and at least one other essential oil.

2. The composition of claim 1, comprising three essential oils.

3. The composition of claim 1, comprising four essential oils.

4. The composition of claim 1, comprising five essential oils.

5. The composition of claim 1, comprising six essential oils.

6. The composition of claim 1, wherein the at least one other essential oil is selected from the group consisting of eucalyptus oil, lemongrass oil, petitgrain oil, Rosmarinum officinalis (rosemary) oil, Thymus vulgaris (thyme) oil, Lavendula augustifolia (lavender) oil, Melaleuca alternifolia (tea tree) oil, Tagete minuta (marigold) oil, Levisticum officinalis (lovage) oil, cinnamon oil, lemon oil, orange oil, grapefruit oil and oil of bergamot.

7. The composition of claim 1, comprising at least 5% Lippia javanica essential oil in combination with at least a pair of essential oils selected from among lemongrass oil and lavender oil; lemongrass oil and tea tree oil; lemongrass oil and rosemary oil; lemongrass oil and thyme oil; lemongrass oil and petitgrain oil; lemongrass oil and lemon oil; lemongrass oil and orange oil; lemongrass oil and grapefruit oil; lemongrass oil and eucalyptus oil; lavender oil and tea tree oil; lavender oil and rosemary oil; lavender oil and thyme oil; lavender oil and petitgrain oil; lavender oil and lemon oil; lavender oil and orange oil; lavender oil and grapefruit oil; lavender oil and eucalyptus oil; tea tree oil and rosemary oil; tea tree oil and thyme oil; tea tree oil and petitgrain oil; tea tree oil and lemon oil; tea tree oil and orange oil; tea tree oil and grapefruit oil; tea tree oil and eucalyptus oil; rosemary oil and thyme oil; rosemary oil and petitgrain oil; rosemary oil and lemon oil; rosemary oil and orange oil; rosemary oil and grapefruit oil; rosemary oil and eucalyptus oil; thyme oil and petitgrain oil; thyme oil and lemon oil; thyme oil and orange oil; thyme oil and grapefruit oil; thyme oil and eucalyptus oil; petitgrain oil and lemon oil; petitgrain oil and orange oil; petitgrain oil and grapefruit oil; petitgrain oil and eucalyptus oil; lemon oil and orange oil; lemon oil and grapefruit oil; lemon oil and eucalyptus oil; orange oil and grapefruit oil; orange oil and eucalyptus oil; and grapefruit oil and eucalyptus oil.

8. The composition of claim 1, comprising at least 5% Lippia javanica essential oil in combination with at least one triplet combination of essential oils selected from among lemongrass oil, Lavendula augustifolia and Melaleuca alternifolia oil; lemongrass oil, Lavendula augustifolia and Rosmarinum officinalis oil; lemongrass oil, Lavendula augustifolia and Thymus vulgaris oil; Lavendula augustifolia, Melaleuca alternifolia oil and Rosmarinum officinalis oil; grapefruit oil, Lavendula augustifolia and Melaleuca alternifolia oil; grapefruit oil, Lavendula augustifolia and Rosmarinum officinalis oil; grapefruit oil, Lavendula augustifolia and Thymus vulgaris oil; orange oil, Lavendula augustifolia and Melaleuca alternifolia oil; orange oil, Lavendula augustifolia and Rosmarinum officinalis oil; orange oil, Lavendula augustifolia and Thymus vulgaris oil; lemon oil, Lavendula augustifolia and Melaleuca alternifolia oil; lemon oil, Lavendula augustifolia and Rosmarinum officinalis oil; lemon oil, Lavendula augustifolia and Thymus vulgaris oil; petitgrain oil, Lavendula augustifolia and Melaleuca alternifolia oil; petitgrain oil, Lavendula augustifolia and Rosmarinum officinalis oil; petitgrain oil, Lavendula augustifolia and Thymus vulgaris oil; eucalyptus oil, Lavendula augustifolia and Melaleuca alternifolia oil; eucalyptus oil, Lavendula augustifolia and Rosmarinum officinalis oil; and eucalyptus oil, Lavendula augustifolia and Thymus vulgaris oil.

9. The composition of claim 1, comprising from about 5% to about 70% Lippia javanica oil.

10. The composition of claim 1, further comprising a carrier.

11. The composition of claim 10, wherein the carrier is a fluid carrier.

12. The composition of claim 11, wherein the fluid carrier comprises an alcohol or a non-essential oil or a combination of an alcohol and a non-essential oil.

13. The composition of claim 12, wherein the non-essential oil is selected from the group consisting of olive oil, soybean oil, safflower oil, sunflower oil, almond oil, walnut oil, jojoba oil, macadamia nut oil, coconut oil, palm oil, sesame oil, rapeseed oil, canola oil, rice bran oil, grapeseed oil, flaxseed oil, corn oil, cocoa butter, cottonseed oil, avocado oil and combinations thereof.

14. The composition of claim 12, wherein the alcohol is selected from among methanol, ethanol, propanol, isopropanol, butanol, sec-butanol, tert-butanol, pentanol and its various isomers, hexanol and its various isomers, and mixtures thereof.

15. The composition of claim 11, wherein the fluid carrier comprises an amine or an amide or an ether or a ketone or a combination thereof.

16. The composition of claim 15, wherein the carrier fluid comprises an ether selected from the group consisting of diethyl ether, n-propyl ether, isopropyl ether, ethylene glycol monomethyl ether, ethylene glycol dimethyl ether, ethylene glycol monoethyl ether, ethylene glycol diethylether, propylene glycol mono-and dimethyl ethers, propylene glycol mono and diethyl ethers, butylene glycol mono and dimethyl ethers, butylene glycol mono and diethyl ethers, and combinations thereof.

17. The composition of claim 15, wherein the carrier fluid comprises a ketone selected from the group consisting of acetone, methyl ethyl ketone, methyl propyl ketone, ethyl ketone, methyl isopropyl ketone, benzyl methyl ketone, and combinations thereof.

18. The composition of claim 11, wherein the fluid carrier is an aerosol propellant that is gaseous at room temperature and pressure.

19. The composition of claim 18, wherein the aerosol propellant is selected from the group consisting of propane, butane, isobutane and carbon dioxide.

20. The composition of claim 10, wherein the carrier is a solid.

21. The composition of claim 20, wherein the solid carrier is a salt crystal, a granule, a dust or a powder.

22. The composition of claim 21, wherein the powder is sodium bicarbonate or an inert dispersible finely divided solid selected from the group consisting of kaolins, clays, vermiculite, alumina, silica, chalk, calcium carbonate, talc, attapulgite, montmorillonite, kieselguhr, silicic acid, amorphous or fumed silica, pyrophyllite, diatomaceous earths, vermiculite, a calcium phosphate, a calcium carbonate and a magnesium carbonate.

23. The composition of claim 1, further comprising a silicone.

24. The composition of claim 23, wherein silicone is selected from the group consisting of dimethicone copolyol, cyclomethicone, polydimethylsiloxane, cyclic dimethyl polysiloxane, aminosilicones, phenylsilicones, diphenyldimethicones, phenyltrimethicones, cyclopentasiloxane, dimethicone copolyol (a polymer of dimethyl-siloxane with polyoxyethylene and/or polyoxypropylene), dimethicone copolyol, cetyldimethicone copolyol, cetyl dimethicone, cetyl dimethiconecopolyol, amino-propyl PEG-7 PEG-3 dimethicone copolyol and a dimethiconol.

25. The composition of claim 1, further comprising an emulsifier.

26. The composition of claim 25, wherein the emulsifier is selected from among stearic acid, cetyl alcohol, PEG-100, stearate and glyceryl stearate, cetearyl glucoside, polysorbate 20, ceteareth-20, cetyl alcohol, cetearyl alcohol, a self-emulsifying wax, isopropyl palmitate, cetyl palmitate, stearyl alcohol, lecithin, hydrogenated lecithin, steareth-2, steareth-20 and polyglyceryl-2 stearate.

27. The composition of claim 1, further comprising a humectant and/or an emollient.

28. The composition of claim 27, wherein the humectant and/or emollient is selected from the group consisting of alkylmonoglycerides, alkyldiglycerides, triglycerides, hydrogenated oils, lanolin derivatives, mineral oils, paraffin oils, perhydrosqualane, squalene, 1,2-propanediol, 1,3-butanediol, cetyl alcohol, stearyl alcohol, oleic alcohol, glycerin, glycol, butylene glycol, polyethylene glycol esters, polyethylene glycols or polypropylene glycols, a fatty ester, isopropyl palmitate, 2-ethylhexyl cocoate, myristyl myristate, isopropyl myristate, isopropyl stearate, glyceryl triacetate, ascorbyl palmitate, octyl dodecanol, hexyl decanol, oleyl alcohol, decyl oleate, hexyl laureate, dioctyl cyclohexane, glycearyl stearate, Ceteareth 20, Cetereath 12, cetyl palmitate, esters of lactic acid, stearic acid, behenic acid, isostearic acid, lecithin, collagen, elastin, keratin, lanolin, sorbitol, xylitol, maltitol, polydextrose, petrolatum, paraffin, benzyl alcohol, butyl alcohol, cetearyl alcohol, cetyl alcohol, myristyl alcohol, diglyceryl caprylate and combinations thereof.

29. The composition of claim 1, further comprising a preservative.

30. The composition of claim 29, wherein the preservative is selected from the group consisting of an ester of p-hydroxybenzoic acid, methylparaben, propylparaben, butylparaben, sodium benzoate, potassium benzoate, sorbic acid, 2-phenoxyethanol, methylchloroisothiazolinone, methylisothiazolinone, hexylene glycol, chlorphenesin or combinations thereof.

31. The composition of claim 1, further comprising an antioxidant.

32. The composition of claim 31, wherein the antioxidant is selected from the group consisting of ascorbic acid, butylated hydroxyl anisole, butylated hydroxy toluene, propyl gallate, tartaric acid, phosphoric acid, erythrobic acid, lactic acid, sodium sulfite, sodium bisulfate, sodium metabisulfite, thioglycolic acid, cysteine hydrochloride, a tocopherol or combinations thereof.

33. The composition of claim 31, wherein the antioxidant is a tocopherol or a blend of tocopherols.

34. The composition of claim 1, further comprising a viscosity-modifying polymer.

35. A method of killing, combating or controlling ectoparasites or pests, comprising:
applying to at least one of (a) such ectoparasites or pests and (b) the locus to be protected, a pesticidally effective amount of the composition of claim 1, whereby the ectoparasite or pest is killed.

36. The method claim 35, wherein the locus to be protected is selected from among a bedding area, a room, carpeting, bedding, clothing, upholstery or a subject.

37. A method for treating a subject infested with ectoparasites and/or pests, comprising the steps of topically applying to the subject a therapeutically effective amount of a composition comprising:
a) at least 5% Lippia javanica oil in combination with one or more essential oils;
b) a non-essential oil; and
c) an alcohol, wherein the composition is applied to the subject for a time sufficient to kill the ectoparasites and/or pests.

38. The method of claim 37, wherein the composition is applied for at least 30 minutes.

39. The method of claim 37, wherein the composition is applied for about 20 minutes.

40. The method of claim 37, wherein the composition is applied for about 10 minutes.

41. The method of claim 37, wherein the composition is applied for about 5 minutes.

42. The method of claim 37, wherein the composition is applied to the hair or skin of the subject so that the hair or the skin is totally saturated with the composition.

43. A method of treating an infestation of ectoparasites and/or pests on a subject, comprising:
(a) topically applying a composition of claim 1 to the infestation; and
(b) allowing the composition to remain on the infestation for at least about five minutes.

44. The method of claim 43, wherein the ectoparasites are selected from the group consisting of biting insects, true bugs, flies, fleas, ticks, mites and lice.

45. The method of claim 44, wherein the lice are selected from the group consisting of head lice, body lice and pubic lice.

46. A method of treating an infestation of nits of head lice, in both mature and immature forms, comprising:
(a) topically applying a composition of claim 1 to the infestation; and
(b) allowing the composition to remain on the infestation for at least about 5 minutes.

47. A method of treating a head lice infestation, in both mature and immature forms, comprising:
(a) topically applying a composition of claim 1 to the infestation; and
(b) allowing the composition to remain on the infestation for at least about 5 minutes.

48. A method of killing ectoparasites and/or pests on a subject, comprising:
providing a non-aqueous composition of claim 1;
applying to an area on the subject where ectoparasites and/or pests are present an amount of the composition sufficient to kill ectoparasites and/or pests when the medicament is applied to an area on the subject where ectoparasites and/or pests are present; and
allowing the composition to remain on the area on the subject where ectoparasites and/or pests are present for at least about 5 minutes.

49. The method of claim 48, wherein the ectoparasites are selected from the group consisting of lice, ticks, mites and fleas.

50. The method of claim 48, wherein the subject is a mammal.

51. The method of claim 50, wherein the mammal is a human and the ectoparasites are head lice.

52. A method of killing ectoparasites and/or pests, comprising:
applying to the ectoparasites and/or pests an amount of the composition of claim 1 sufficient to kill ectoparasites and/or pests when the composition is applied to the ectoparasites and/or pests; and
allowing the composition to remain on the ectoparasites and/or pests until the ectoparasites and/or pests are dead.

53. The method of claim 52, wherein the ectoparasites and/or pests are selected from the group consisting of flies, fleas, spiders, ticks, mites, lice, ants, no-see-ums, wall lice, horse lice, chinch bugs, redcoats, bedbugs, chiggers, sand fleas, snow fleas, human fleas, mosquitoes, drain flies, mites, weevils, millipedes, centipedes, carpet beetles, gnats, fruit flies, cluster flies, phorid flies and black flies.

54. A method of repelling ectoparasites and/or pests, comprising:
applying topically to a subject or a locus in which ectoparasites and/or pests are to be repelled an amount of the composition of claim 1 effective to repel but not kill ectoparasites or pests.

55. The method of claim 54, wherein the ectoparasites and/or pests are selected from the group consisting of flies, fleas, spiders, ticks, mites, lice, ants, no-see-ums, chinch bugs, redcoats, bedbugs, chiggers, sand fleas, mosquitoes, drain flies, mites, weevils, millipedes, centipedes, carpet beetles, gnats, fruit flies, cluster flies, phorid flies and black flies.

56. The method of claim 54, wherein the ectoparasites are mosquitoes, lice, ticks, mites or fleas.

57. The method of claim 54, wherein the ectoparasites are head lice, body lice or pubic lice.

58. A method of repelling ectoparasites and/or pests, comprising:
atomizing or dispersing the composition of claim 1 as a fine spray or mist in the atmosphere of a locus where the ectoparasites and/or pests are to be repelled in an amount sufficient to repel but not kill the ectoparasites and/or pests.

59. The method of claim 58, wherein the composition of claim 1 is dispersed in the atmosphere using a spray pump or aerosol.

60. The method of claim 58, wherein the composition of claim 1 is dispersed in the atmosphere using a diffuser.

61. An article of manufacture, comprising:
a packaging material;
within the packaging material an essential-oil composition of claim 1; and
a label that indicates that the essential oil composition of claim 1 is used for repelling or killing an ectoparasite and/or pest.

62. An article of manufacture, comprising:
an essential-oil composition of claim 1; and
a container for dispensing the essential oil composition.

63. The article of manufacture of claim 62, further comprising instructions for use of the composition.

64. A kit, comprising:
a composition of claim 1 in a package or other enclosure; and
a flea comb or a nit comb to assist in removing ectoparasites and/or pests and their eggs from hair.

65. A kit, comprising:
a composition of claim 1; and
a diffuser to dispense the composition into the atmosphere.

66. A kit, comprising:
a composition of claim 1;
a testing assay for the presence of ectoparasites and/or pests; and
instructions for using the composition to kill ectoparasites and/or pests.

67. The kit of claim 66, wherein the assay is a guanine test for the presence of mites.
